SEQUENCE LISTING (16 sequences)

SEQ ID NO: 1 - DNA, 33 bp, Artificial Sequence
  the sequence is synthesized
tttccctctagaccggcaatagttacccttatt

SEQ ID NO: 2 - DNA, 32 bp, Artificial Sequence
  the sequence is synthesized
tgacagagatctcattttgtttattgcaattg

SEQ ID NO: 3 - DNA, 32 bp, Artificial Sequence
  the sequence is synthesized
aaaatgagatctctgtcagaccaagtttactc

SEQ ID NO: 4 - DNA, 30 bp, Artificial Sequence
  the sequence is synthesized
tgccggtctagagggaaaccgttgtggtct

SEQ ID NO: 5 - DNA, 29 bp, Artificial Sequence
  the sequence is synthesized
aagtaagaattctgctagcagaagcttcg

SEQ ID NO: 6 - DNA, 33 bp, Artificial Sequence
  the sequence is synthesized
ggggcactcgaggggttagtgacattagaaaac

SEQ ID NO: 7 - DNA, 32 bp, Artificial Sequence
  the sequence is synthesized
atcctactcgagtgccccgttagttgaagaag

SEQ ID NO: 8 - DNA, 35 bp, Artificial Sequence
  the sequence is synthesized
tagcatgaattcaaacgatcccgcgaaattaatac

SEQ ID NO: 9 - DNA, 29 bp, Artificial Sequence
  the sequence is synthesized
aagtaagaattctgctagcagaagcttcg

SEQ ID NO: 10 - DNA, 35 bp, Artificial Sequence
  the sequence is synthesized
agatctggatccaaacgatcccgcgaaattaatac

SEQ ID NO: 11 - DNA, 34 bp, Artificial Sequence
  the sequence is synthesized
ggatccagatctgaatatttcagcttggttttcc

SEQ ID NO: 12 - DNA, 42 bp, Artificial Sequence
  the sequence is synthesized
agctaagaattcgctacgatggatccttcctcctttaattgg

SEQ ID NO: 13 - DNA, 33 bp, Artificial Sequence
  the sequence is synthesized
gaggaaggatccatgttggagttcgaaacaaac

SEQ ID NO: 14 - DNA, 34 bp, Artificial Sequence
  the sequence is synthesized
actgcccgaaccgcttccgccgcgtttattacgg

SEQ ID NO: 15 - DNA, 35 bp, Artificial Sequence
  the sequence is synthesized
ggaagcggttcgggcagtatggtgagcaagggcga

SEQ ID NO: 16 - DNA, 31 bp, Artificial Sequence
  the sequence is synthesized
ctagcagaattcttacttgtacagctcgtcc

(19) United States
(12) Patent Application Publication
(10) Pub. No.: US 2017/0348258 A1
(43) Pub. Date: Dec. 7, 2017

(54) 3-Amino Benzamide Derivative Serving As B-Lactam Auxiliary Antibiotic, Preparation Method and Use Thereof

(71) Applicant: THE HONG KONG POLYTECHNIC UNIVERSITY, Hong Kong (HK)

(72) Inventors: Kwok-Yin Wong, Hong Kong (HK); Tak-Hang Chan, Hong Kong (HK); Kin-Fai Chan, Hong Kong (HK); Sheng Chen, Hong Kong (HK); Hok-Kiu Lui, Hong Kong (HK); Jason Wing-Yiu Kan, Hong Kong (HK); Jia-Chi Chiou, Hong Kong (HK); Hong-Kin Yap, Hong Kong (HK)

(21) Appl. No.: 15/509,145

(22) PCT Filed: Sep. 9, 2015
(86) PCT No.: PCT/CN2015/089286

§ 371 Date: Jul. 20, 2017

(30) Foreign Application Priority Data
Sep. 9, 2014 (CN) 201410456500.6

(51) Int. Cl. A61K 31/166

(52) U.S. Cl. CPC A61K 31/166

(57) ABSTRACT

This invention discloses 3-aminobenzamide derivatives as β-lactam antibiotic adjuvants of chemical formula (I), processes for preparing them and their use. The derivatives can act as β-lactam antibiotic adjuvants for the treatment of methicillin-resistant staphylococcal infections.

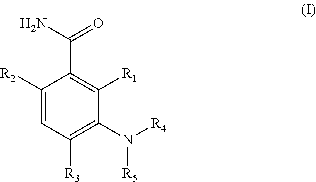

### CROSS-REFERENCE TO RELATED APPLICATIONS

This application claims priority from PCT Application No. PCT/CN2015/089286, filed Sep. 9, 2015, which in turn claims priority from Chinese Patent Application No. 201410456500.6, filed Sep. 9, 2014, the disclosures of which are incorporated by reference in their entirety.

### TECHNICAL FIELD

The present invention pertains to the field of pharmaceutical technology and relates to 3-aminobenzamide derivatives, which can be used to treat methicillin-resistant staphylococcal infections, to processes for preparing them and their use in the preparation of β-lactam antibiotic adjuvants.

### BACKGROUND

Staphylococcus aureus is the most widespread bacterial pathogen in the developed countries. Serious bacterial infections caused by such pathogen, especially multidrug-resistant Staphylococcus aureus (MRSA) have caused considerable patient mortality worldwide. Nowadays, more than two-thirds of S. aureus are resistant to methicillin, a second generation of β-lactam antibiotic. Alarmingly, clinical bacterial strains that are resistant even to vancomycin hydrochloride, which is the antibiotic of last resort against MRSA, have emerged recently. In 2005, about 95,000 people acquired MRSA infections in the United States, of which nearly 19,000 people died. In Hong Kong, MRSA has also emerged as one of the most important pathogens, in particular community-acquired MRSA (CA-MRSA). The number of notifications for CA-MRSA has been rising rapidly from Year 2007 (82 cases) to Year 2013 (988 cases), which is about 12-fold increase within seven years. Among those various notifiable infectious diseases, CA-MRSA infections represent one of the top four serious infectious diseases in Hong Kong. More importantly, according to a report published in 2007 by Centre for Health Protection, Department of Health, HKSAR, two patients died because of CA-MRSA infection. Considering the emergence of MRSA and limited clinical antibiotic treatment options, there is an urgent need to develop new drugs to fight against MRSA infection.

### SUMMARY

The technical problem to be solved by the present invention is to provide a 3-aminobenzamide derivative for use as a β-lactam antibiotic adjuvant against the deficiencies of lack of drugs effective in treating MRSA infection in the prior art.

A further problem to be solved by the present invention is to provide a simple and efficient process for the preparation of 3-aminobenzamide derivatives.

Another problem to be solved by the present invention is to provide use of the above derivatives in the preparation of β-lactam antibiotic adjuvants.

The technical scheme employed by the present invention in the solution of its technical problems is a 3-aminobenzamide derivative as a β-lactam antibiotic adjuvant, which is a compound of the following chemical formula (I),

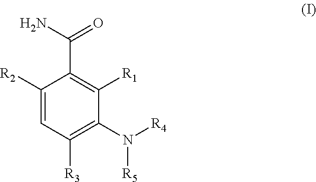

wherein:
R₁, R₂ and R₃ each respectively are hydrogen, fluorine or bromine;
when R₄ and R₅ are different, R₄ is hydrogen, methyl, ethyl or fluorobenzyl, and R₅ is C₄-C₁₀ alkyl, alkoxyalkyl, C₄-C₁₀ alkenyl, fluorobenzyl, benzyl substituted by alkoxy, or acyl;
when R₄ and R₅ are the same, R₄ and R₅ are both CH₂—(CH₂)ₙ—CH₂, and n=2 or 3.

In the 3-aminobenzamide derivatives, preferably, R₁, R₂, R₃ and R₄ each respectively are hydrogen, and R₅ is octyl. The derivative is 3-(octylamino)benzamide.

In the 3-aminobenzamide derivatives, preferably, R₁, R₂, R₃ and R₄ each respectively are hydrogen, and R₅ is nonyl. The derivative is 3-(nonylamino)benzamide.

In the 3-aminobenzamide derivatives, preferably, R₁, R₂ and R₃ each respectively are hydrogen, R₄ is methyl, and R₅ is octyl. The derivative is 3-(methyl(octyl)amino)benzamide.

In the 3-aminobenzamide derivatives, preferably, R₁, R₂ and R₃ each respectively are hydrogen, R₄ is methyl, and R₅ is nonyl. The derivative is 3-(methyl(nonyl)amino)benzamide.

In the 3-aminobenzamide derivatives, preferably, R₁ and R₃ each respectively are hydrogen, R₂ is F, R₄ is hydrogen, and R₅ is nonyl. The derivative is 2-fluoro-5-(nonylamino)benzamide.

In the 3-aminobenzamide derivatives, preferably, R₁ and R₃ each respectively are hydrogen, R₂ and R₄ each are F, and R₅ is nonyl. The derivative is 2,4-difluoro-5-(nonylamino)benzamide.

The Technical Scheme of the First Preparation Method:

the preparation method of the above derivatives, comprising the steps of:

(a) mixing the starting material A with the starting material B and potassium carbonate in acetonitrile and stirring well to form a reaction mixture, heating the reaction mixture to reflux for 4 hours, and then refining to obtain the product; wherein the starting material A is 3-aminobenzamide, 2-fluoro-5-aminobenzamide or 2,4-difluoro-5-aminobenzamide, the starting material B is 1-bromononane or 1-bromooctane;
(b) when the product obtained in step (a) is 3-(nonylamino)benzamide or 3-(octylamino)benzamide, stirring it well with dimethyl sulfate and potassium carbonate in acetonitrile respectively, heating to reflux for 12 hours, and then refined to obtain the product. The above preparation method is shown as follows.

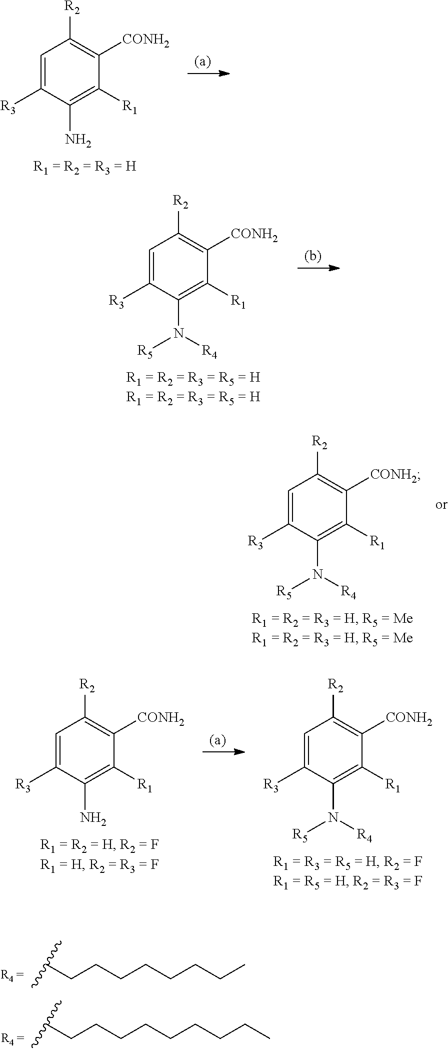

In the 3-aminobenzamide derivatives, preferably, R₁ and R₂ are F, R₃ and R₄ each respectively are hydrogen, and R₅ is octyl. The derivative is 2,6-difluoro-3-(octylamino)benzamide.

In the 3-aminobenzamide derivatives, preferably, R₁ and R₂ are F, R₃ and R₄ each are hydrogen, and R₅ is nonyl. The derivative is 2,6-difluoro-3-(nonylamino)benzamide.

In the 3-aminobenzamide derivatives, preferably, R₁ and R₂ are F, R₃ and R₄ each are hydrogen, and R₅ is heptyl. The derivative is 2,6-difluoro-3-(heptylamino)benzamide.

In the 3-aminobenzamide derivatives, preferably, R₁ and R₂ are F, R₃ and R₄ each are hydrogen, and R₅ is 3,7-dimethyl-2,6-octadien-1-yl. The derivative is (E)-3-((3,7-dimethyl-2,6-octadien-1-yl)amino)-2,6-difluorobenzamide.

In the 3-aminobenzamide derivatives, preferably, R¹ and R² are F, R³ and R⁴ each are hydrogen, and R⁵ is 2-nonenyl. The derivative is (Z)-2,6-difluoro-3-(2-nonenylamino)benzamide.

In the 3-aminobenzamide derivatives, preferably, R₁ and R₂ are F, R₃ and R₄ each are hydrogen, and R₅ is (4′-butoxy)butyl. The derivative is 3-((4′-butoxy)butylamino)-2,6-difluorobenzamide.

In the 3-aminobenzamide derivatives, preferably, R₁ and R₂ are F, R₃ and R₄ each are hydrogen, and R₅ is decyl. The derivative is 3-(decylamino)-2,6-difluorobenzamide.

In the 3-aminobenzamide derivatives, preferably, R₁ and R₂ are F, R₃ is bromine, R₄ is hydrogen, and R₅ is nonyl. The derivative is 4-bromo-2,6-difluoro-3-(nonylamino)benzamide.

In the 3-aminobenzamide derivatives, preferably, R₁ and R₂ are F, R₃ is hydrogen, R₄ is methyl, and R₅ is octyl. The derivative is 2,6-difluoro-3-(methyl(octyl)amino)benzamide.

In the 3-aminobenzamide derivatives, preferably, R₁ and R₂ are F, R₃ is hydrogen, R₄ is ethyl, and R₅ is octyl. The derivative is 3-(ethyl(octyl)amino)-2,6-difluorobenzamide.

In the 3-aminobenzamide derivatives, preferably, R₁ and R₂ are F, R₃ is hydrogen, R₄ is methyl, and R₅ is nonyl. The derivative is 2,6-difluoro-3-(methyl(nonyl)amino)benzamide.

The Technical Scheme of the Second Preparation Method:

the preparation method of the above derivatives, comprising the steps of:

(a) mixing the starting material 2,6-difluoro-3-aminobenzamide with brominated alkanes and potassium carbonate in acetonitrile and stirring well to form a reaction mixture, heating the reaction mixture to reflux for 4 hours, and then refining to obtain the product; the brominated alkanes are 1-bromononane, 1-bromooctane, 1-bromodecane, geranyl bromide, (Z)-1-bromonon-2-ene or 1-bromoheptane; (b) brominating the 2,6-difluoro-3-(nonylamino)benzamide obtained by the reaction of step (a) and stirring at room temperature for 12 hours, and then refining to obtain 4-bromo-2,6-difluoro-3-(nonylamino)benzamide;
(c) further alkylating the 2,6-difluoro-3-(octylamino)benzamide obtained from step (a) using methyl iodide or bromoethane, mixing with potassium carbonate in acetonitrile with stirring under reflux for 14 hours, and then refining to obtain 2,6-difluoro-3-(methyl(octyl)amino)benzamide or 3-(ethyl(octyl)amino)-2,6-difluorobenzamide;
(d) alkylating the 2,6-difluoro-3-(nonylamino)benzamide obtained from step (a) using dimethyl sulfate, mixing with potassium carbonate in acetonitrile with stirring under reflux for 12 hours, and then refining to obtain 2,6-difluoro-3-(methyl(nonyl)amino)benzamide. The above preparation method is shown as follows.

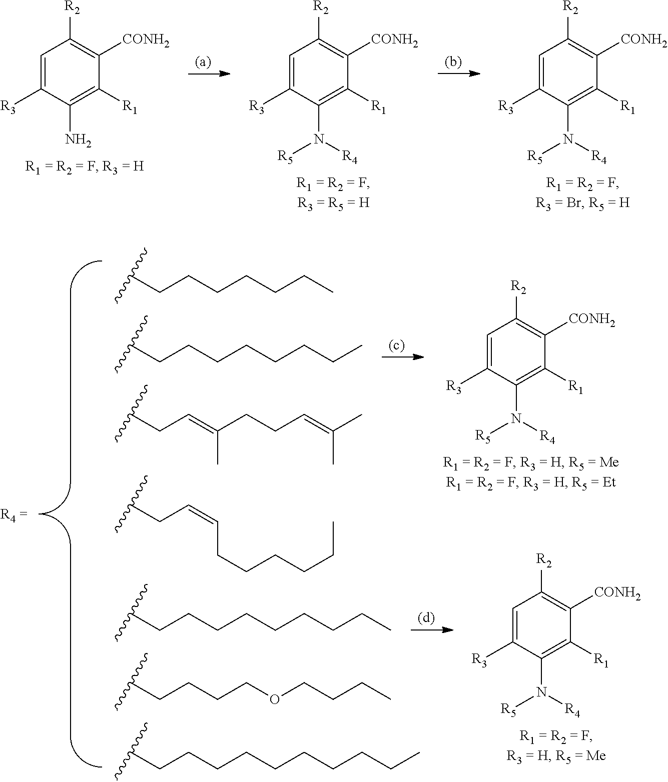

In the 3-aminobenzamide derivatives, preferably, R₁ and R₂ are F, R₃ and R₄ each are hydrogen, and R₅ is nonanamido. The derivative is 2,6-difluoro-3-nonanamidobenzamide.

In the 3-aminobenzamide derivatives, preferably, R₁ and R₂ are F, R₃ and R₄ each are hydrogen, and R₅ is pyrrolidine. The derivative is 2,6-difluoro-3-(pyrrolidin-1-yl)benzamide.

In the 3-aminobenzamide derivatives, preferably, R₁ and R₂ are F, R₃ and R₄ each are hydrogen, and R₅ is piperidine. The derivative is 2,6-difluoro-3-(piperidin-1-yl)benzamide.

In the 3-aminobenzamide derivatives, preferably, R₁ and R₂ are F, R₃ and R₄ each are hydrogen, and R₅ is 3′,4′-difluorobenzyl. The derivative is 3-(3′,4′-difluorobenzylamino)-2,6-difluorobenzamide.

In the 3-aminobenzamide derivatives, preferably, R₁ and R₂ are F, R₃ is hydrogen, R₄ is 3′,4′-difluorobenzyl, and R₅ is 3′,4′-difluorobenzyl. The derivative is 3-(bis(3′,4′-difluorobenzyl)amino)-2,6-difluorobenzamide.

In the 3-aminobenzamide derivatives, preferably, R₁ and R₂ are F, R₃ and R₄ each are hydrogen, and R₅ is 2′,4′-difluorobenzyl. The derivative is 3-(2′,4′-difluorobenzylamino)-2,6-difluorobenzamide.

In the 3-aminobenzamide derivatives, preferably, R₁ and R₂ are F, R₃ is hydrogen, R₄ and R₅ each are 2′,4′-difluorobenzyl. The derivative is 3-(bis(2′,4′-difluorobenzyl)amino)-2,6-difluorobenzamide.

In the 3-aminobenzamide derivatives, preferably, R₁ and R₂ are F, R₃ and R₄ each are hydrogen, and R₅ is 2′,6′-difluorobenzyl. The derivative is 3-(2′,6′-difluorobenzylamino)-2,6-difluorobenzamide.

In the 3-aminobenzamide derivatives, preferably, R₁ and R₂ are F, R₃ is hydrogen, R₄ and R₅ each are 2′,6′-difluorobenzyl. The derivative is 3-(bis(2′,6′-difluorobenzyl)amino)-2,6-difluorobenzamide.

The Technical Scheme of the Third Preparation Method:

the preparation method of the above derivatives, comprising the steps of:

(a) dissolving the starting materials 2,6-difluoro-3-aminobenzamide and nonanoyl chloride in pyridine and dichloromethane at 0° C. and allowing to react for 4 hours, and refining to obtain 2,6-difluoro-3-nonylaminobenzamide;
(b) adding potassium carbonate to the starting materials 2,6-difluoro-3-aminobenzamide and 1,4-dibromobutane or 1,5-dibromopentane in acetonitrile respectively and stirring well for 4 hours, and then refining to obtain 2,6-difluoro-3-(pyrrolidin-1-yl)benzamide and 2,6-difluoro-3-(piperidin-1-yl)benzamide, respectively;
(c) heating the starting material 2,6-difluoro-3-aminobenzamide and the starting material C and potassium carbonate in acetonitrile to reflux for 4 hours, and refining to obtain 3-(3′,4′-difluorobenzylamino)-2,6-difluorobenzamide or 3-(bis(3′,4′-difluorobenzyl)amino)-2,6-difluorobenzamide, 3-(2′,4′-difluorobenzylamino)-2,6-difluorobenzamide or 3-(bis(2′,4′-difluorobenzyl)amino)-2,6-difluorobenzamide, and 3-(2′,6′-difluorobenzylamino)-2,6-difluorobenzamide or 3-(bis(2′,6′-difluorobenzyl)amino)-2,6-difluorobenzamide, respectively; whererin the starting material C is 3,4-difluorobenzyl bromide, 2,4-difluorobenzyl bromide or 2,6-difluorobenzyl bromide. The above preparation method is shown as follows.

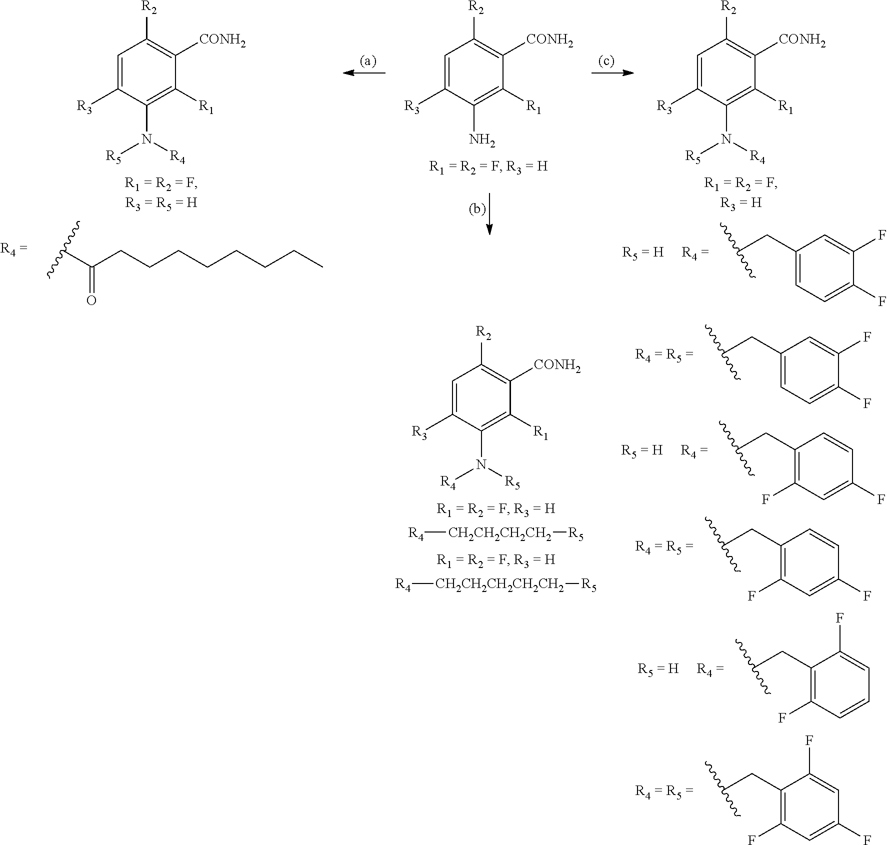

In the 3-aminobenzamide derivatives, preferably, R₁ and R₂ are F, R₃ and R₄ each are hydrogen, and R₅ is 3′-butoxybenzyl. The derivative is 3-((3′-butoxybenzyl)amino)-2,6-difluorobenzamide.

In the 3-aminobenzamide derivatives, preferably, R₁ and R₂ are F, R₃ and R₄ each are hydrogen, and R₅ is 3′-(sec-butoxy)benzyl. The derivative is 3-((3′-(sec-butoxy)benzyl)amino)-2,6-difluorobenzamide.

In the 3-aminobenzamide derivatives, preferably, R₁ and R₂ are F, R₃ and R₄ each are hydrogen, and R₅ is 3′-(pentyloxy)benzyl. The derivative is 2,6-difluoro-3-((3′-(pentyloxy)benzyl)amino)benzamide.

In the 3-aminobenzamide derivatives, preferably, R₁ and R₂ are F, R₃ and R₄ each are hydrogen, and R₅ is 3′-((4″-(trifluoromethyl)benzyl)oxy)benzyl. The derivative is 2,6-difluoro-3-((3′-((4″-(trifluoromethyl)benzyl)oxy)benzyl)amino)benzamide.

The Technical Scheme of the Fourth Preparation Method:

the preparation method of the above derivatives, comprising the steps of:

(a) mixing the starting material 2,6-difluoro-3-aminobenzamide with the corresponding benzaldehydes and the catalyst p-toluenesulfonic acid (pTsOH) in methanol, respectively, and stirring the mixture at room temperature for 2 hours;
(b) then adding sodium cyanoborohydride in portions, mixing and stirring for 12 hours to carry out reductive amination, and refining to obtain the above derivatives respectively; wherein the benzaldehydes are 3-butoxybenzaldehyde, 3-(pentyloxy)benzaldehyde, 3-(sec-butoxy)benzaldehyde or 3-((4′-(trifluoromethyl)benzyl)oxy)benzaldehyde. The above preparation method is shown as follows.

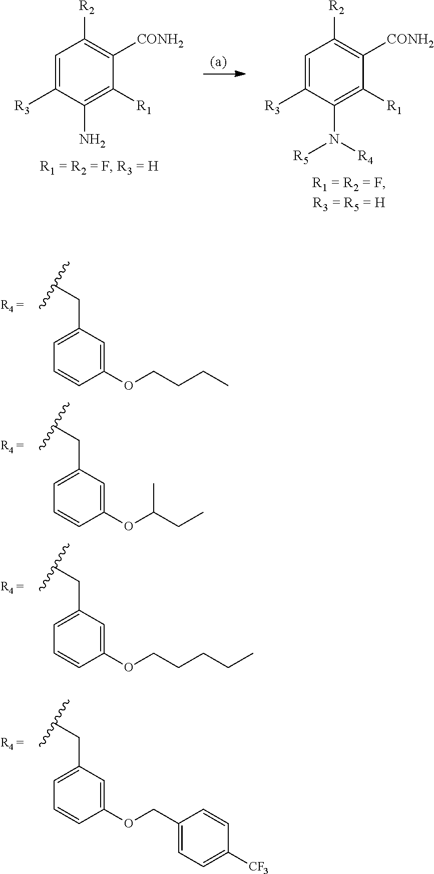

In addition, the present invention discloses a use of the above derivatives in the preparation of β-lactam antibiotic adjuvants.

The derivatives at oral dosage of 50 mg/Kg body weight were orally bioavailable at 12% bioavailability.

Bacterial cell division machinery has attracted considerable attention from academia and pharmaceutical industry recently for exploring potential drug targets of novel antibacterial agents. Among those identified targets so far, filamenting temperature-sensitive mutant Z (FtsZ) represents the most promising and well-characterized one. FtsZ is a guanosine triphosphatase and is highly conserved in a wide range of bacteria. For bacteria to initiate cell division, FtsZ monomers initially localize to mid-cell and polymerize into single stranded straight protofilaments via head-to-tail association in a guanosine triphosphate (GTP) dependent manner. Lateral contacts between single stranded FtsZ protofilaments result in the formation of FtsZ bundles and eventually leading to Z-ring formation. Subsequent recruitment of other downstream proteins responsible for the invagination of cell membrane and septum formation completes the cell division process. With such an important role in bacterial cell division, FtsZ has therefore been recognized as an attractive target for the development of novel antibacterial agents. Several high resolution X-ray crystal structures were solved for a variety of FtsZ homologs and revealed a very important conserved general organization comprising two independent folding domains (FIG. 1a). The N-terminal domain forms the guanosine triphosphate binding site (GTP binding site), while C-terminal domain contains a flexible loop (T7 loop). Both domains were interconnected via a long central helix 7 (H7). A dozen of FtsZ-interacting compounds have been reported in the literature either binding to the GTP binding site or T7 loop, some of which have powerful antibacterial activity with minimum inhibition concentration (MIC) at micromolar range.

The present invention discloses a novel compound (I) derived from 3-aminobenzamide (FIG. 1b, Formula 4), which has the lowest minimal inhibitory concentration (MIC) on S. aureus in an antibacterial susceptibility test, and has low toxicity on mouse fibroblasts in the cytotoxicity test. These two derivatives with potent antibacterial activity were further subjected to biological assays, such as light scattering assay, GTPase activity assay, cell morphology study and fluorescence microscopy. All results clearly indicated that these two derivatives interact with the cell division protein FtsZ. We also demonstrated that such derivatives in combination with clinically used β-lactam antibiotics exhibited highly synergic antibacterial activity against clinically isolated community associated S. aureus (USA300) and other MRSA strains. Pharmacokinetics study revealed that compound F332 at oral dosage of 50 mg/Kg body weight was orally bioavailable at about 12% bioavailability, supporting its use in the preparation of β-lactam antibiotic adjuvants and the treatment of methicillin-resistant staphylococcal infections by oral route, that is, the compounds of the present invention can be used clinically as β-lactam antibiotic adjuvants to treat methicillin-resistant S. aureus infections by oral route.

### BRIEF DESCRIPTION OF THE FIGURES

The present invention will now be further illustrated with reference to the accompanying drawings and the following examples.

FIG. 1a shows the crystal structure of the S. aureus FtsZ.

FIG. 1b shows the chemical structural formula of PC190723, and the basic structures of its derivatives (formulas 1, 2, 3) and 3-aminobenzamide (formula 4).

FIG. 2 shows the first preparation method disclosed in the embodiments of the present invention.

FIG. 3 shows the second preparation method disclosed in the embodiments of the present invention.

FIG. 4 shows the third preparation method disclosed in the embodiments of the present invention.

FIG. 5 shows the fourth preparation method disclosed in the embodiments of the present invention.

FIG. 6 shows an electron microscopy image of the Bacillus subtilis 168 cells grown in dimethyl sulfoxide solution for 4 hours in accordance with the embodiments of the present invention.

FIG. 7 shows an electron microscopy image of the B. subtilis 168 cells grown in 1.56 μM F332 solution for 4 hours.

FIG. 8 shows an electron microscopy image of the B. subtilis 168 cells grown in 0.78 μM F361 solution for 4 hours.

FIG. 9 shows an SDS-PAGE gel diagram of the S. aureus FtsZ (lanes 4-8).

FIG. 10a shows the structural map of the expression plasmid of the B. subtilis FtsZ-eGFP fusion protein.

FIG. 10b shows a fluorescence microscopy image of B. subtilis 168 transformed by the expression plasmid of the FtsZ-eGFP fusion protein and incubated for 4 hours without any effect of the derivatives of the present invention.

FIG. 10c shows a phase contrast microscopy image.

FIG. 10d shows a fluorescence microscopy image of B. subtilis 168 transformed by the expression plasmid of the FtsZ-eGFP fusion protein and incubated in the presence of 3 μM F332 for 4 hours.

FIG. 10e shows a phase contrast microscopy image.

FIG. 11 shows a graph of the effect of F332 on FtsZ polymerization in the S. aureus FtsZ (5 μM).

FIG. 12 shows a graph of the effect of F361 on FtsZ polymerization in the S. aureus FtsZ (5 μM).

FIG. 13 shows the effect of F332 on GTPase activity of the S. aureus FtsZ.

FIG. 14 shows the effect of F361 on GTPase activity of the S. aureus FtsZ.

FIG. 15 shows an electron micrograph of the FtsZ polymer.

FIG. 16 shows an electron micrograph of the FtsZ polymer

FIG. 17 shows the pharmacokinetic parameters of F332.

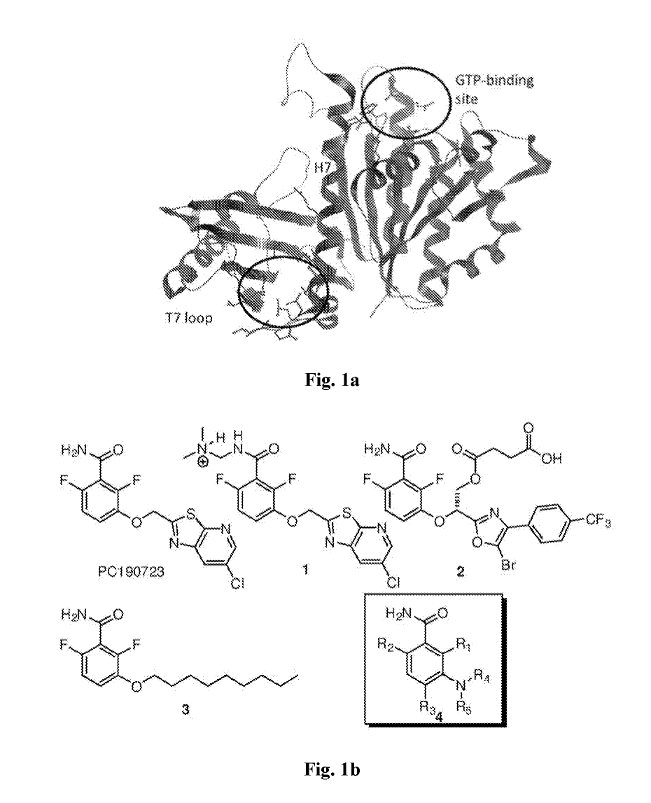

### DETAILED DESCRIPTION

For a clear understanding of the technical features, objects and effects of the present invention, specific embodiments of the present invention will now be described in detail with reference to the accompanying drawings.

In the present invention, the preparation of the 3-aminobenzamide derivatives is divided into four cases according to different starting materials and products, and the following four preparation methods are listed respectively.

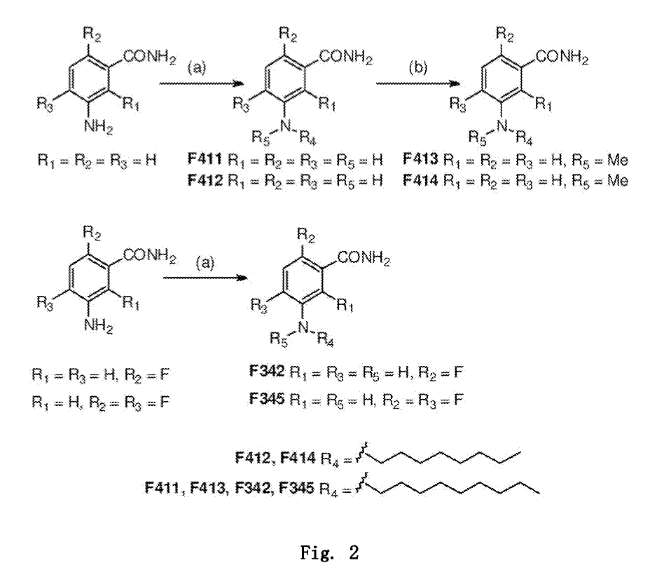

### The First Preparation Method: Synthesis of Compounds F411, F412, F413, F414, F342 and F345 (See the Corresponding Compounds as Numbered in FIG. 2), Corresponding to the Following Examples 1-6

The synthetic steps were shown in FIG. 2.

Commercially available 3-aminobenzamide is mixed with 1-bromononane or 1-bromooctane in acetonitrile (ACN) under basic conditions for refluxing temperature to produce F411 and F412 respectively.

These two compounds were further alkylated to tertiary amine by using dimethyl sulfate under basic conditions to afford F413 and F414.

Similarly, commercially available 2-fluoro-5-aminobenzamide or 2,4-difluoro-5-aminobenzamide is mixed with 1-bromononane in ACN under basic conditions at refluxing temperature to afford F342 and F345 respectively.

The experimental procedures were shown in FIG. 2, which included the following steps:

(a) mixing the starting material A with the starting material B and potassium carbonate in acetonitrile and stirring well to form a reaction mixture, and heating the reaction mixture to reflux for 4 hours to obtain the product; wherein the starting material A is 3-aminobenzamide, 2-fluoro-5-aminobenzamide or 2,4-difluoro-5-aminobenzamide; the starting material B is 1-bromononane or 1-bromooctane;
(b) when the product obtained in step (a) is 3-(nonylamino)benzamide or 3-(octylamino)benzamide, stirring it well with dimethyl sulfate and potassium carbonate in acetonitrile respectively, and heating to reflux for 12 hours to obtain the product.

### Example 1: 3-(nonylamino)benzamide (F411)

To a well stirred solution of 3-aminobenzamide (0.20 g, 1.4 mmol) and 1-bromononane (0.32 g, 1.5 mmol) in acetonitrile (20 mL) was added K₂CO₃ (0.23 g, 1.6 mmol). The reaction mixture was heated to reflux for 4 hrs. After the complete disappearance of starting material as indicated by thin layer chromatography (TLC), the reaction mixture was subjected to pass through a short pad of silica gel. The filtrate obtained was evaporated under reduced pressure and subjected to flash column chromatography using silica gel. The titled compound (0.15 g) was obtained in 39% yield: ¹HNMR (400 MHz, CHLOROFORM-d) δ 7.23 (t, J=7.83 Hz, 1H), 7.11 (t, J=1.96 Hz, 1H), 7.03 (d, J=7.34 Hz, 1H), 6.75 (dd, J=2.20, 7.58 Hz, 1H), 6.15 (br. s., 1H), 5.99 (br. s., 1H), 3.15 (t, J=7.09 Hz, 2H), 1.63 (quin, J=7.21 Hz, 2H), 1.25-1.45 (m, 12H), 0.84-0.95 (m, 3H); ¹³C NMR (101 MHz, CHLOROFORM-d) δ 170.2, 148.8, 134.4, 129.3, 116.1, 115.3, 111.6, 43.9, 31.9, 29.6, 29.4, 29.3, 27.1, 22.7, 14.1; LRMS (ESI) m/z 263 (M⁺+H, 100), 285 (M⁺+Na, 8); HRMS (ESI) calcd for C₁₆H₂₇N₂O (M⁺+H) 263.2123, found 263.2112.

### Example 2: 3-(octylamino)benzamide (F412)

To a well stirred solution of 3-aminobenzamide (0.20 g, 1.4 mmol) and 1-bromooctane (0.29 g, 1.5 mmol) in acetonitrile (20 mL) was added K₂CO₃ (0.23 g, 1.6 mmol). The reaction mixture was heated to reflux for 4 hrs. After the complete disappearance of starting material as indicated by TLC, the reaction mixture was subjected to pass through a short pad of silica gel. The filtrate obtained was evaporated under reduced pressure and subjected to flash column chromatography using silica gel. The titled compound (0.18 g) was obtained in 47% yield: ¹HNMR (400 MHz, CHLOROFORM-d) δ 7.23 (t, J=7.83 Hz, 1H), 7.07-7.18 (m, 1H), 6.97-7.07 (m, 1H), 6.75 (dd, J=2.20, 7.58 Hz, 1H), 6.15 (br. s., 1H), 5.98 (br. s., 1H), 3.15 (t, J=7.09 Hz, 2H), 1.64 (quin, J=7.21 Hz, 2H), 1.22-1.47 (m, 10H), 0.91 (t, J=6.85 Hz, 3H); ¹³C NMR (101 MHz, CHLOROFORM-d) δ 170.1, 148.8, 134.4, 129.3, 116.1, 115.3, 111.6, 43.9, 31.8, 29.4, 29.4, 29.3, 27.1, 22.7, 14.1; LRMS (ESI) m/z 249 (M⁺+H, 100), 271 (M⁺+Na, 10); HRMS (ESI) calcd for C₁₅H₂₅N₂O (M⁺+H) 249.1967, found 249.1957.

### Example 3: 3-(methyl(nonyl)amino)benzamide (F413)

To a well stirred solution of 3-(nonylamino)benzamide (0.09 g, 0.3 mmol) and dimethyl sulfate (0.06 g, 0.5 mmol) in ACN (10 mL) was added K₂CO₃ (0.06 g, 0.4 mmol). The reaction mixture was heated to reflux for 12 hrs. After the complete disappearance of starting material as indicated by TLC, the reaction mixture was diluted with ethyl acetate (20 mL) and subjected to pass through a short pad of silica gel. The filtrate obtained was evaporated under reduced pressure and subjected to flash column chromatography using silica gel. The titled compound (0.04 g) was obtained in 42% yield: ¹HNMR (400 MHz, CHLOROFORM-d) δ 7.23-7.32 (m, 1H), 7.21 (s, 1H), 6.99 (d, J=7.34 Hz, 1H), 6.84 (dd, J=2.45, 8.31 Hz, 1H), 6.15 (br. s., 1H), 5.95 (br. s., 1H), 3.30-3.41 (m, 2H), 2.96 (s, 3H), 1.53-1.64 (m, 2H), 1.22-1.37 (m, 12H), 0.83-0.95 (m, 3H); ¹³C NMR (101 MHz, CHLOROFORM-d) δ 170.5, 149.5, 134.3, 129.2, 115.3, 113.8, 111.1, 52.7, 38.4, 31.9, 29.6, 29.5, 29.3, 27.1, 26.7, 22.7, 14.1; LRMS (ESI) m/z 277 (M⁺+H, 100), 299 (M⁺+Na, 7); HRMS (ESI) calcd for C₁₇H₂₉N₂O (M⁺+H) 277.2280, found 277.2271.

### Example 4: 3-(methyl(octyl)amino)benzamide (F414)

To a well stirred solution of 3-(octylamino)benzamide (0.08 g, 0.3 mmol) and dimethyl sulfate (0.05 g, 0.4 mmol) in ACN (10 mL) was added K₂CO₃ (0.06 g, 0.4 mmol). The reaction mixture was heated to reflux for 12 hrs. After the complete disappearance of starting material as indicated by TLC, the reaction mixture was diluted with ethyl acetate (20 mL) and subjected to pass through a short pad of silica gel. The filtrate obtained was evaporated under reduced pressure and subjected to flash column chromatography using silica gel. The titled compound (0.04 g) was obtained in 47% yield: ¹HNMR (400 MHz, CHLOROFORM-d) δ 7.23-7.32 (m, 1H), 7.21 (s, 1H), 6.99 (d, J=7.82 Hz, 1H), 6.84 (dd, J=2.45, 8.31 Hz, 1H), 6.14 (br. s., 1H), 5.97 (br. s., 1H), 3.29-3.41 (m, 2H), 2.98 (s, 3H), 1.52-1.66 (m, 2H), 1.23-1.39 (m, 10H), 0.85-0.96 (m, 3H); ¹³C NMR (101 MHz, CHLOROFORM-d) δ 170.5, 149.5, 134.3, 129.2, 115.3, 113.8, 111.1, 52.7, 38.4, 31.8, 29.5, 29.3, 27.2, 26.7, 22.7, 14.1; LRMS (ESI) m/z 263 (M⁺+H, 100), 285 (M⁺+Na, 10); HRMS (ESI) calcd for C₁₆H₂₇N₂O (M⁺+H) 263.2123, found 263.2113.

### Example 5: 2-fluoro-5-(nonylamino)benzamide (F342)

To a well stirred solution of 2-fluoro-5-aminobenzamide (0.20 g, 1.3 mmol) and 1-bromononane (0.30 g, 1.4 mmol) in ACN (20 mL) was added K₂CO₃ (0.25 g, 1.8 mmol). The reaction mixture was heated to reflux for 4 hrs. After the complete disappearance of starting material as indicated by TLC, the reaction mixture was subjected to pass through a short pad of silica gel. The filtrate obtained was evaporated under reduced pressure and subjected to flash column chromatography using silica gel. The titled compound (0.11 g) was obtained in 30% yield: ¹H NMR (400 MHz, CHLOROFORM-d) δ 7.23-7.34 (m, 1H), 6.94 (d, J=8.80 Hz, 1H), 6.76 (s, 1H), 6.62-6.71 (m, 1H), 6.28 (br. s., 1H), 3.70 (br. s., 1H), 3.11 (t, J=7.09 Hz, 2H), 1.61 (quin, J=7.09 Hz, 2H), 1.22-1.44 (m, 12H), 0.89 (t, J=6.60 Hz, 3H); ¹³C NMR (101 MHz, CHLOROFORM-d) δ 165.6, 154.8, 152.5, 145.4, 145.4, 120.2, 120.0, 117.5, 117.4, 116.7, 116.4, 114.3, 44.4, 31.9, 29.5, 29.4, 29.3, 27.1, 22.7, 14.1; LRMS (ESI) m/z 281 (M⁺+H, 100), 303 (M⁺+Na, 50); HRMS (ESI) calcd for C₁₆H₂₆N₂OF (M⁺+H) 281.2029, found 281.2033.

### Example 6: 2,4-difluoro-5-(nonylamino)benzamide (F345)

To a well stirred solution of 2,4-difluoro-5-aminobenzamide (0.20 g, 1.1 mmol) and 1-bromononane (0.28 g, 1.4 mmol) in ACN (20 mL) was added K₂CO₃ (0.23 g, 1.7 mmol). The reaction mixture was heated to reflux for 4 hrs. After the complete disappearance of starting material as indicated by TLC, the reaction mixture was subjected to pass through a short pad of silica gel. The obtained filtrate was evaporated under reduced pressure and subjected to flash column chromatography using silica gel. The titled compound (0.09 g) was obtained in 26% yield: ¹H NMR (400 MHz, CHLOROFORM-d) δ 7.54-7.34 (m, 1H), 6.75-6.92 (m, 1H), 6.60-6.75 (m, 1H), 6.23 (br. s., 1H), 3.79 (br. s., 1H), 3.45-3.09 (m, 2H), 1.56-1.71 (m, 2H), 1.19-1.44 (m, 14H), 0.80-0.96 (m, 3H); LRMS (ESI) m/z 299 (M⁺+H, 100), 321 (M⁺+Na, 85); ¹³C NMR (101 MHz, CHLOROFORM-d) δ 164.9, 162.7, 154.1, 153.5, 151.5, 151.1, 134.3, 134.1, 131.7, 131.6, 118.6, 115.9, 113.4, 113.3, 104.0, 103.9, 103.6, 103.6, 103.3, 77.4, 77.2, 77.1, 76.7, 43.8, 40.1, 31.9, 29.5, 29.5, 29.4, 29.4, 29.3, 29.3, 29.2, 27.0, 27.0, 22.7, 14.1; LRMS (ESI) m/z 299 (M⁺+H, 100), 321 (M⁺+Na, 85); HRMS (ESI) calcd for C₁₆H₂₅N₂OF₂ (M⁺+H) 299.1935, found 299.1939.

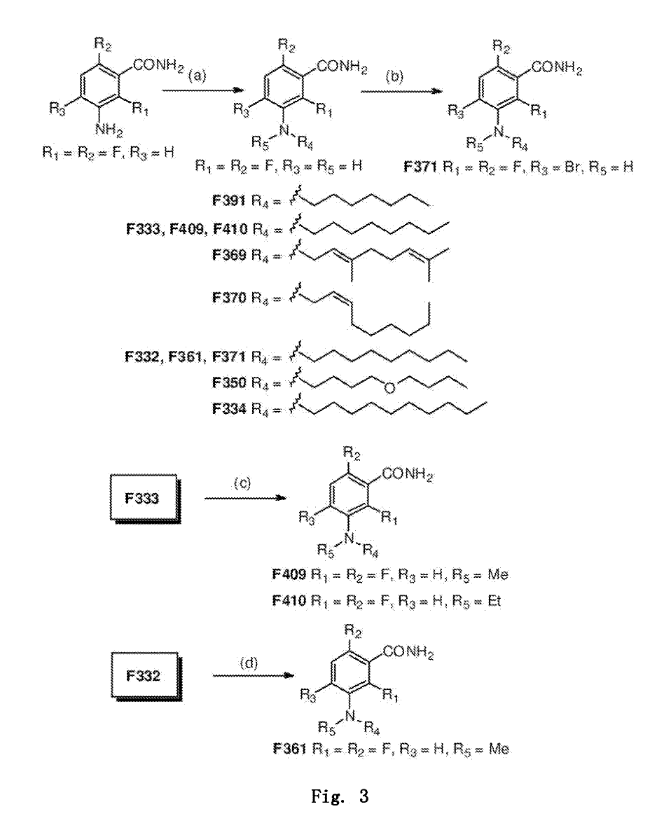

### The Second Preparation Method: Synthesis of Compounds F332, F333, F334, F350, F361, F369, F370, F371, F391, F409 and F410 (See the Corresponding Compounds as Numbered in FIG. 3), Corresponding to the Following Examples 7-18

The synthetic procedures of the compounds F332, F333, F334, F350, F361, F369, F370, F371, F391, F409 and F410 of the present invention are shown in FIG. 3.

Large scale synthesis of 2,6-difluoro-3-aminobenzamide was carried out in two steps from commercially available 2,6-difluoro-3-nitrobenzonic acid.

Firstly, treatment of 2,6-difluoro-3-nitrobenzonic acid with thionyl chloride followed by aqueous ammonia solution furnished 2,6-difluoro-3-nitrobenzamide, which was further converted into 2,6-difluoro-3-aminobenzamide by using tin (II) chloride in conc. hydrochloric acid.

Then, treatment of 2,6-difluoro-3-aminobenzamide with various alkyl halides, such as 1-bromononane, 1-bromooctane, 1-bromodecane, 1-bromo-4-butoxybutane, geranyl bromide, (Z)-1-bromonon-2-ene and 1-bromoheptane, in ACN under basic conditions for 4 hours furnished F332, F333, F334, F350, F369, F370 and F391 respectively.

Bromination of F332 furnished F371.

Further alkylation of F332 and F333 by using dimethyl sulfate or bromoethane afforded F409, F410 and F361.

The detailed experimental procedures were shown in FIG. 3. (a) various brominated alkanes, K₂CO₃, ACN, reflux, 4 hrs; (b) Br², DCM, r.t., 12 hrs; (c) methyl iodide or bromoethane, K₂CO₃, ACN, reflux, 14 hrs; (d) dimethyl sulfate, K₂CO₃, ACN, reflux, 12 hrs.

### Example 7: 2,6-difluoro-3-aminobenzamide

A well stirred mixture of 2,6-difluoro-3-nitrobenzonic acid (44 g, 216 mmol) and thionyl chloride (100 mL) was heated to reflux for 2 hrs. Excess thionyl chloride was removed by evaporation to afford 2,6-difluoro-3-nitrobenzonic acid chloride, which was used in the next step without further purification. To a well stirred 30% aqueous 30% ammonia solution (300 mL) at 0° C. was added 2,6-difluoro-3-nitrobenzonic acid chloride dropwise. After the addition, the precipitate formed was collected by filtration to afford 2,6-difluoro-3-nitrobenzamide (40 g, 91%), which was used in the next step without further purification. To a well stirred solution of tin (II) chloride (80 g, 421 mmol) in conc. HCl (200 mL) at 0° C. was added 2,6-difluoro-3-nitrobenzamide in portions. After the addition, the reaction mixture was stirred at room temperature for 12 hrs. The reaction was quenched by adding to the reaction mixture excess potassium hydroxide solution until the pH was >12 at 0° C. The aqueous solution was extracted with ethyl acetate (200 mL×4). The combined organic layers were dried over anhydrous magnesium sulfate (MgSO₄), filtered and evaporated to dryness to give 2,6-difluoro-3-aminobenzamide (18 g, 53%): ¹H NMR (400 MHz, Acetone) δ 7.36 (br. s., 1H), 7.14 (br. s., 1H), 6.87 (dt, J=5.38, 9.29 Hz, 1H), 6.77 (t, J=9.05 Hz, 1H), 4.67 (br. s., 2H); ¹³C NMR (101 MHz, Acetone) δ 162.3, 151.7, 151.7, 149.3, 148.2, 148.1, 145.8, 145.7, 133.0, 132.9, 132.8, 132.8, 116.4, 116.4, 116.3, 116.3, 115.5, 115.3, 115.3, 115.1, 111.0, 110.9, 110.7, 110.7.

### Example 8: 2,6-difluoro-3-(nonylamino)benzamide (F332)

To a well stirred solution of 2,6-difluoro-3-aminobenzamide (0.74 g, 4.3 mmol) and 1-bromononane (1.20 g, 5.8 mmol) in ACN (50 mL) was added K₂CO₃ (1.20 g, 8.7 mmol) and catalytic amount of NaI (0.08 g). The reaction mixture was heated to reflux for 4 hrs. After the complete disappearance of starting material as indicated by TLC, the reaction mixture was subjected to pass through a short pad of silica gel. The filtrate obtained was evaporated under reduced pressure and subjected to flash column chromatography using silica gel. The titled compound (0.49 g) was obtained in 38% yield: ¹H NMR (400 MHz, CHLOROFORM-d) δ 6.77-6.94 (m, 1H), 6.69 (dt, J=5.38, 9.29 Hz, 1H), 6.12 (br. s., 1H), 6.05 (br. s., 1H), 3.82 (br. s., 1H), 3.12 (t, J=7.09 Hz, 2H), 1.58-1.73 (m, 2H), 1.23-1.46 (m, 12H), 0.90 (t, J=6.85 Hz, 3H); ¹³C NMR (101 MHz, CHLOROFORM-d) δ 162.8, 151.9, 149.5, 149.2, 146.8, 134.2, 134.1, 113.1, 113.0, 111.4, 111.4, 111.2, 111.2, 43.9, 31.9, 29.5, 29.4, 29.3, 29.2, 27.0, 22.7, 14.1; LRMS (ESI) m/z 299 (M⁺+H, 97), 321 (M⁺+Na, 100); HRMS (ESI) calcd for C₁₆H₂₄N₂OF₂Na (M⁺+Na) 321.1754, found 321.1756.

### Example 9: 2,6-difluoro-3-(octylamino)benzamide (F333)

To a well stirred solution of 2,6-difluoro-3-aminobenzamide (0.40 g, 2.3 mmol) and 1-bromooctane (0.45 g, 2.3 mmol) in ACN (20 mL) was added K₂CO₃ (0.40 g, 2.9 mmol) and catalytic amount of NaI (0.04 g). The reaction mixture was heated to reflux for 4 hrs. After the complete disappearance of starting material as indicated by TLC, the reaction mixture was subjected to pass through a short pad of silica gel. The filtrate obtained was evaporated under reduced pressure and subjected to flash column chromatography using silica gel. The titled compound (0.26 g) was obtained in 39% yield: ¹H NMR (400 MHz, CHLOROFORM-d) δ 6.83 (t, J=9.29 Hz, 1H), 6.68 (dt, J=5.38, 9.05 Hz, 1H), 6.36 (br. s., 1H), 6.09 (br. s., 1H), 3.81 (br. s., 1H), 3.06-3.17 (m, 2H), 1.59-1.69 (m, 2H), 1.23-1.43 (m, 10H), 0.90 (t, J=6.60 Hz, 3H); ¹³C NMR (101 MHz, CHLOROFORM-d) δ 163.0, 151.9, 149.5, 149.5, 149.2, 149.1, 146.7, 134.2, 134.2, 134.1, 134.1, 113.1, 113.1, 113.0, 113.0, 112.5, 111.4, 111.4, 111.2, 111.2, 43.9, 31.9, 31.8, 29.4, 29.3, 29.2, 27.0, 22.6, 14.1; LRMS (ESI) m/z 285 (M⁺+H, 100), 307 (M⁺+Na, 20); HRMS (ESI) calcd for C₁₅H₂₃N₂OF₂ (M⁺+H) 285.1778, found 285.1773.

### Example 10: 3-(decylamino)-2,6-difluorobenzamide (F334)

To a well stirred solution of 2,6-difluoro-3-aminobenzamide (0.40 g, 2.3 mmol) and 1-bromodecane (0.56 g, 2.5 mmol) in ACN (20 mL) was added K₂CO₃ (0.40 g, 2.9 mmol) and catalytic amount of NaI (0.04 g). The reaction mixture was heated to reflux for 4 hrs. After the complete disappearance of starting material as indicated by TLC, the reaction mixture was subjected to pass through a short pad of silica gel. The filtrate obtained was evaporated under reduced pressure and subjected to flash column chromatography using silica gel. The titled compound (0.27 g) was obtained in 37% yield: ¹H NMR (400 MHz, CHLOROFORM-d) δ 6.77-6.89 (m, 1H), 6.68 (dt, J=5.14, 9.17 Hz, 1H), 6.29 (br. s., 1H), 6.10 (br. s., 1H), 3.75-3.89 (m, 1H), 3.12 (q, J=6.52 Hz, 2H), 1.59-1.68 (m, 2H), 1.21-1.49 (m, 14H), 0.90 (t, J=6.36 Hz, 3H); ¹³C NMR (101 MHz, CHLOROFORM-d) δ 163.3, 162.9, 134.2, 134.1, 113.1, 113.0, 113.0, 112.9, 111.4, 111.4, 111.2, 111.1, 57.9, 43.9, 42.3, 31.9, 31.9, 29.6, 29.6, 29.6, 29.5, 29.4, 29.3, 29.3, 27.7, 27.3, 27.0, 22.7, 14.1; LRMS (ESI) m/z 313 (M⁺+H, 28), 335 (M⁺+Na, 95); HRMS (ESI) calcd for C₁₇H₂₆N₂OF₂Na (M⁺+Na) 335.1911, found 335.1923.

### Example 11: 3-((4′-butoxy)butylamino)-2,6-difluorobenzamide (F350)

To a well stirred solution of 2,6-difluoro-3-aminobenzamide (0.17 g, 1.0 mmol) and 1-bromo-4-butoxybutane (0.21 g, 1.0 mmol) in ACN (20 mL) was added K₂CO₃ (0.15 g, 1.1 mmol). The reaction mixture was heated to reflux for 4 hrs. After the complete disappearance of starting material as indicated by TLC, the reaction mixture was subjected to pass through a short pad of silica gel. The filtrate obtained was evaporated under reduced pressure and subjected to flash column chromatography using silica gel. The titled compound (0.05 g) was obtained in 17% yield: ¹H NMR (400 MHz, CHLOROFORM-d) δ 6.75-6.93 (m, 1H), 6.68 (dt, J=5.38, 9.29 Hz, 1H), 6.32 (br. s., 1H), 6.09 (br. s., 1H), 3.95 (br. s., 1H), 3.44 (td, J=6.17, 12.59 Hz, 4H), 3.16 (br. s., 2H), 1.65-1.76 (m, 4H), 1.53-1.61 (m, 2H), 1.32-1.44 (m, 2H), 0.93 (t, J=7.34 Hz, 3H); ¹³C NMR (101 MHz, CHLOROFORM-d) δ 163.0, 151.9, 146.7, 134.1, 113.1, 113.0, 113.0, 112.9, 111.4, 111.4, 111.1, 70.8, 70.3, 43.7, 31.8, 27.2, 26.2, 19.4, 13.9; LRMS (ESI) m/z 301 (M⁺+H, 40); HRMS (ESI) calcd for C₁₅H₂₃N₂O₂F₂ (M⁺+H) 301.1728, found 301.1716.

### Example 12: 2,6-difluoro-3-(methyl(nonyl)amino)benzamide (F361)

To a well stirred solution of 2,6-difluoro-3-(nonylamino)benzamide (0.15 g, 0.5 mmol) and dimethyl sulfate (0.15 g, 1.2 mmol) in acetone (20 mL) was added K₂CO₃ (0.15 g, 1.1 mmol). The reaction mixture was heated to reflux for 14 hrs. After the complete disappearance of starting material as indicated by TLC, the reaction mixture was subjected to pass through a short pad of silica gel. The filtrate obtained was evaporated under reduced pressure and subjected to flash column chromatography using silica gel. The titled compound (0.03 g) was obtained in 19% yield: ¹H NMR (400 MHz, CHLOROFORM-d) δ 6.90-7.02 (m, 1H), 6.79-6.90 (m, 1H), 6.39 (br. s., 1H), 6.05 (br. s., 1H), 3.00-3.08 (m, 2H), 2.79 (s, 3H), 1.53 (br. s., 2H), 1.27 (br. s., 12H), 0.89 (t, J=6.60 Hz, 3H); ¹³C NMR (101 MHz, CHLOROFORM-d) δ 163.0, 154.9, 153.5, 152.4, 137.6, 121.2, 121.1, 116.9, 111.2, 111.2, 111.0, 55.6, 55.5, 40.0, 40.0, 31.9, 29.6, 29.5, 29.3, 27.2, 27.0, 22.7, 14.1; LRMS (ESI) m/z 313 (M⁺+H, 100); HRMS (ESI) calcd for C₁₇H₂₇N₂OF₂ (M⁺+H) 313.2091, found 313.2083.

### Example 13

### (E)-3-((3,7-dimethyl-2,6-octadien-1-yl)amino)-2,6-difluorobenzamide (F369)

To a well stirred solution of 2,6-difluoro-3-aminobenzamide (0.34 g, 2.0 mmol) and geranyl bromide (0.42 g, 2.0 mmol) in ACN (20 mL) was added K₂CO₃ (0.29 g, 2.1 mmol). The reaction mixture was heated to reflux for 4 hrs. After the complete disappearance of starting material as indicated by TLC, the reaction mixture was subjected to pass through a short pad of silica gel. The filtrate obtained was evaporated under reduced pressure and subjected to flash column chromatography using silica gel. The titled compound (0.29 g) was obtained in 48% yield: ¹HNMR (400 MHz, CHLOROFORM-d) δ 6.83 (dt, J=1.47, 9.29 Hz, 1H), 6.68 (dt, J=5.38, 9.05 Hz, 1H), 6.48 (br. s., 1H), 6.11 (br. s., 1H), 5.30 (t, J=6.11 Hz, 1H), 5.03-5.13 (m, 1H), 3.83 (br. s., 1H), 3.72 (d, J=6.85 Hz, 2H), 2.02-2.16 (m, 4H), 1.66-1.75 (m, 6H), 1.62 (s, 3H); ¹³C NMR (101 MHz, CHLOROFORM-d) δ 163.1, 152.1, 149.7, 146.8, 146.8, 139.9, 134.1, 133.9, 131.8, 123.8, 120.7, 113.4, 113.4, 113.3, 113.3, 111.4, 111.2, 111.1, 41.8, 39.5, 26.3, 25.7, 17.7, 16.4; LRMS (ESI) m/z 309 (M⁺+H, 100), 321 (M⁺+Na, 6); HRMS (ESI) calcd for C₁₇H₂₃N₂OF₂ (M⁺+H) 309.1778, found 309.1779.

### Example 14: (Z)-2,6-difluoro-3-(2-nonenylamino)benzamide (F370)

To a well stirred solution of 2,6-difluoro-3-aminobenzamide (0.17 g, 1.0 mmol) and (Z)-1-bromonon-2-ene (0.21 g, 1.0 mmol) in ACN (20 mL) was added K₂CO₃ (0.15 g, 1.1 mmol). The reaction mixture was heated to reflux for 4 hrs. After the complete disappearance of starting material as indicated by TLC, the reaction mixture was subjected to pass through a short pad of silica gel. The filtrate obtained was evaporated under reduced pressure and subjected to flash column chromatography using silica gel. The titled compound (0.13 g) was obtained in 44% yield: ¹H NMR (400 MHz, CHLOROFORM-d) δ 6.84 (dt, J=1.47, 9.29 Hz, 1H), 6.70 (dt, J=5.38, 9.29 Hz, 1H), 6.28 (br. s., 1H), 6.08 (br. s., 1H), 5.52-5.61 (m, 1H), 5.32-5.43 (m, 1H), 3.88 (br. s., 1H), 3.09-3.18 (m, 2H), 2.40 (q, J=6.85 Hz, 2H), 2.06 (q, J=7.17 Hz, 2H), 1.24-1.41 (m, 6H), 0.90 (t, J=6.85 Hz, 3H); ¹³C NMR (101 MHz, CHLOROFORM-d) δ 162.9, 159.0, 152.0, 149.6, 149.2, 133.9, 133.4, 125.4, 113.3, 113.2, 113.2, 113.1, 111.4, 111.2, 111.2, 43.4, 31.5, 29.3, 27.3, 27.2, 27.0, 22.5, 14.0, 14.0; LRMS (ESI) m/z 297 (M⁺+H, 100), 319 (M⁺+Na, 35); HRMS (ESI) calcd for C₁₆H₂₃N₂OF₂ (M⁺+H) 297.1778, found 297.1768.

### Example 15: 4-bromo-2,6-difluoro-3-(nonylamino)benzamide (F371)

To a well stirred solution of 2,6-difluoro-3-(nonylamino)benzamide (0.3 g, 1.0 mmol) in dichloromethane (20 mL) at room temperature was added excess bromine (1 mL) and stirred for 12 hrs. After the complete disappearance of starting material as indicated by TLC, the reaction mixture was poured into a separating funnel containing saturated sodium thiosulfate solution (30 mL) and extracted with ethyl acetate (20 mL×3). The combined organic layers were dried over MgSO₄, filtered and evaporated to give a crude product which was further subjected to purification by flash column chromatography to afford the titled compound (0.28 g, 74%): ¹HNMR (400 MHz, CHLOROFORM-d) δ 7.11 (dd, J=1.96, 8.80 Hz, 1H), 6.76 (br. s., 1H), 6.19 (br. s., 1H), 3.74 (br. s., 1H), 3.28 (t, J=6.11 Hz, 2H), 1.52-1.61 (m, 2H), 1.24-1.38 (m, 12H), 0.83-0.92 (m, 3H); ¹³C NMR (101 MHz, CHLOROFORM-d) δ 162.4, 152.9, 152.8, 150.9, 150.9, 150.4, 150.3, 148.3, 133.0, 133.0, 132.9, 132.9, 115.8, 115.7, 115.5, 115.5, 114.9, 114.9, 114.8, 114.7, 113.9, 113.7, 113.5, 47.3, 47.2, 31.9, 31.6, 30.7, 29.5, 29.3, 29.2, 26.8, 22.7, 14.2, 14.1; LRMS (ESI) m/z 377 (M⁺+H, 96), 399 (M⁺+Na, 16); HRMS (ESI) calcd for C₁₆H₂₄N₂OF₂Br (M⁺+H) 377.1040, found 377.1049.

### Example 16: 2,6-difluoro-3-(heptylamino)benzamide (F391)

To a well stirred solution of 2,6-difluoro-3-aminobenzamide (0.70 g, 4.1 mmol) and 1-bromoheptane (0.80 g, 4.4 mmol) in ACN (50 mL) was added K₂CO₃ (0.60 g, 4.4 mmol) and catalytic amount of NaI (0.08 g). The reaction mixture was heated to reflux for 4 hrs. After the complete disappearance of starting material as indicated by TLC, the reaction mixture was subjected to pass through a short pad of silica gel. The filtrate obtained was evaporated under reduced pressure and subjected to flash column chromatography using silica gel. The titled compound (0.40 g) was obtained in 36% yield: ¹H NMR (400 MHz, CHLOROFORM-d) δ 6.85 (dt, J=1.96, 9.29 Hz, 1H), 6.69 (dt, J=5.38, 9.29 Hz, 1H), 6.14 (br. s., 1H), 6.05 (br. s., 1H), 3.12 (t, J=7.09 Hz, 2H), 1.59-1.69 (m, 2H), 1.27-1.45 (m, 8H), 0.86-0.96 (m, 3H); ¹³C NMR (101 MHz, CHLOROFORM-d) δ 166.4, 154.2, 149.6, 145.8, 140.8, 134.2, 113.1, 111.5, 111.2, 105.2, 43.9, 31.8, 29.3, 29.1, 27.0, 22.6, 14.1; LRMS (ESI) m/z 271 (M⁺+H, 100), 293 (M⁺+Na, 60); HRMS (ESI) calcd for C₁₄H₂₁N₂OF₂ (M⁺+H) 271.1622, found 271.1612.

### Example 17: 2,6-difluoro-3-(methyl(octyl)amino)benzamide (F409)

To a well stirred solution of 2,6-difluoro-3-(octylamino)benzamide (0.12 g, 0.4 mmol) and methyl iodide (0.30 g, 2.1 mmol) in ACN (10 mL) was added K₂CO₃ (0.30 g, 2.1 mmol). The reaction mixture was heated to reflux for 14 hrs. After the complete disappearance of starting material as indicated by TLC, the reaction mixture was subjected to pass through a short pad of silica gel. The filtrate obtained was evaporated under reduced pressure and subjected to flash column chromatography using silica gel. The titled compound (0.03 g) was obtained in 24% yield: ¹HNMR (400 MHz, CHLOROFORM-d) δ 6.95 (dt, J=5.62, 9.17 Hz, 1H), 6.80-6.90 (m, 1H), 6.63 (br. s., 1H), 6.11 (br. s., 1H), 2.99-3.07 (m, 2H), 2.79 (s, 3H), 1.47-1.58 (m, 2H), 1.22-1.35 (m, 10H), 0.89 (t, J=6.85 Hz, 3H); ¹³C NMR (101 MHz, CHLOROFORM-d) δ 163.2, 154.9, 154.9, 153.6, 152.4, 151.0, 137.5, 121.2, 121.1, 121.1, 114.1, 113.9, 111.2, 111.2, 111.0, 111.0, 55.6, 55.5, 40.0, 31.8, 29.5, 29.3, 27.2, 27.0, 22.6, 14.1; LRMS (ESI) m/z 299 (M⁺+H, 100), 321 (M⁺+Na, 26); HRMS (ESI) calcd for C₁₆H₂₅N₂OF₂ (M⁺+H) 299.1935, found 299.1928.

### Example 18: 3-(ethyl(octyl)amino)-2,6-difluorobenzamide (F410)

To a well stirred solution of 2,6-difluoro-3-(octylamino)benzamide (0.12 g, 0.4 mmol) and bromoethane (0.30 g, 2.7 mmol) in ACN (10 mL) was added K₂CO₃ (0.30 g, 2.1 mmol). The reaction mixture was heated to reflux for 14 hrs. After the complete disappearance of starting material as indicated by TLC, the reaction mixture was subjected to pass through a short pad of silica gel. The filtrate obtained was evaporated under reduced pressure and subjected to flash column chromatography using silica gel. The titled compound (0.04 g) was obtained in 30% yield: ¹HNMR (400 MHz, CHLOROFORM-d) δ 6.95 (dd, J=5.62, 9.05 Hz, 2H), 6.76-6.86 (m, 1H), 6.20 (br. s., 1H), 3.12 (q, J=7.01 Hz, 2H), 3.03 (t, J=7.58 Hz, 2H), 1.43 (br. s., 2H), 1.24 (br. s., 10H), 1.02 (t, J=7.09 Hz, 3H), 0.86 (t, J=6.60 Hz, 3H); ¹³C NMR (101 MHz, CHLOROFORM-d) δ 163.4, 155.3, 154.7, 152.8, 152.8, 152.1, 135.5, 123.6, 123.5, 114.3, 114.1, 113.9, 111.1, 111.0, 110.8, 110.8, 52.3, 52.3, 47.2, 47.2, 31.8, 29.4, 29.2, 27.3, 27.1, 22.6, 14.0, 12.4; LRMS (ESI) m/z 313 (M⁺+H, 100), 335 (M⁺+Na, 16); HRMS (ESI) calcd for C₁₇H₂₇N₂OF₂ (M⁺+H) 313.2091, found 313.2097.

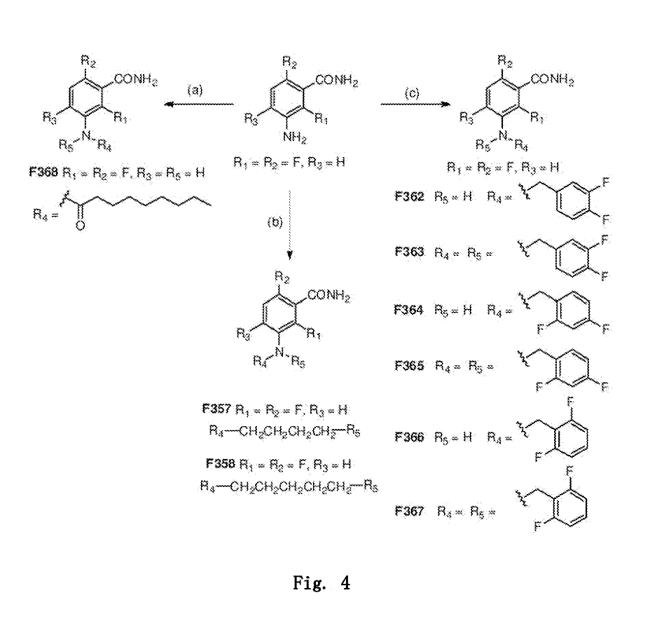

### The Third Preparation Method: Synthesis of Compounds F357, F358, and F362 to F368 (See the Corresponding Compounds as Numbered in FIG. 4), Corresponding to the Following Examples 19-24

The chemical synthesis of the compounds F357, F358, and F362 to F368 of the present invention were shown in FIG. 4. Compound F368 was prepared from 2,6-difluoro-3-aminobenzamide and nonanoyl chloride in pyridine and dichloromethane at 0° C. for 4 hours as shown in FIG. 4.

F357 and F358 were synthesized from 2,6-difluoro-3-aminobenzamide, which refluxed with 1,5-dibromopentane and 1,4-dibromobutane respectively in the presence of potassium carbonate in ACN for 4 hours. F362, F363, F364, F365, F366, F367 were synthesized from 2,6-difluoro-3-aminobenzamide, which refluxed with 3,4-difluorobenzyl bromide, 2,4-difluorobenzyl bromide, 2,6-difluorobenzyl bromide in the presence of potassium carbonate in ACN for 4 hours.

The detailed experimental procedures were shown in FIG. 4. (a) nonanoyl chloride, Py/DCM, 0° C., 4 hrs; (b) 1,5-dibromopentane or 1,4-dibromobutane, K₂CO₃, ACN, reflux, 4 hrs; (c) various difluorobenzyl bromides, K₂CO₃, ACN, reflux, 4 hrs.

### Example 19: 2,6-difluoro-3-(pyrrolidin-1-yl)benzamide (F357)

To a well stirred solution of 2,6-difluoro-3-aminobenzamide (0.17 g, 1.0 mmol) and 1,4-dibromobutane (0.23 g, 1.1 mmol) in ACN (20 mL) was added K₂CO₃ (0.18 g, 1.3 mmol). The reaction mixture was heated to reflux for 4 hrs. After the complete disappearance of starting material as indicated by TLC, the reaction mixture was subjected to pass through a short pad of silica gel. The filtrate obtained was evaporated under reduced pressure and subjected to flash column chromatography using silica gel. The titled compound (0.11 g) was obtained in 46% yield: ¹H NMR (400 MHz, CHLOROFORM-d) δ 6.82 (t, J=8.80 Hz, 1H), 6.69 (dt, J=5.38, 9.29 Hz, 1H), 6.13 (br. s., 1H), 5.99 (br. s., 1H), 3.30-3.41 (m, 4H), 1.97 (td, J=3.48, 6.24 Hz, 4H); ¹³C NMR (101 MHz, CHLOROFORM-d) δ 163.5, 156.9, 155.9, 152.4, 150.9, 138.2, 121.6, 116.5, 116.3, 111.3, 50.0, 50.0, 25.1; LRMS (ESI) m/z 227 (M⁺+H, 100), 249 (M⁺+Na, 57); HRMS (ESI) calcd for C₁₁H₁₃N₂OF₂ (M⁺+H) 227.0996, found 227.1005.

### Example 20: 2,6-difluoro-3-(piperidin-1-yl)benzamide (F358)

To a well stirred solution of 2,6-difluoro-3-aminobenzamide (0.17 g, 1.0 mmol) and 1,5-dibromopentane (0.25 g, 1.1 mmol) in ACN (20 mL) was added K₂CO₃ (0.18 g, 1.3 mmol). The reaction mixture was heated to reflux for 4 hrs. After the complete disappearance of starting material as indicated by TLC, the reaction mixture was subjected to pass through a short pad of silica gel. The filtrate obtained was evaporated under reduced pressure and subjected to flash column chromatography using silica gel. The titled compound (0.10 g) was obtained in 38% yield: ¹H NMR (400 MHz, CHLOROFORM-d) δ 7.00 (dt, J=5.87, 9.05 Hz, 1H), 6.80-6.94 (m, 1H), 6.36 (br. s., 1H), 6.08 (br. s., 1H), 2.91-3.02 (m, 4H), 1.69-1.80 (m, 4H), 1.49-1.64 (m, 2H); ¹³C NMR (101 MHz, CHLOROFORM-d) δ 162.9, 153.2, 151.6, 138.4, 121.1, 121.1, 114.0, 111.3, 111.2, 52.5, 52.4, 26.1, 24.0; LRMS (ESI) m/z 241 (M⁺+H, 100), 263 (M⁺+Na, 30); HRMS (ESI) calcd for C₁₂H₁₅N₂OF₂ (M⁺+H) 241.1152, found 241.1149.

### Example 21: 3-((3′,4′-difluorobenzyl)amino)-2,6-difluorobenzamide (F362) and 3-(bis(3′,4′-difluorobenzyl)amino)-2,6-difluorobenzamide (F363)

To a well stirred solution of 2,6-difluoro-3-aminobenzamide (0.40 g, 2.3 mmol) and 3,4-difluorobenzyl bromide (0.60 g, 2.9 mmol) in ACN (20 mL) was added K₂CO₃ (0.42 g, 3.0 mmol). The reaction mixture was heated to reflux for 4 hrs. After the complete disappearance of starting material as indicated by TLC, the reaction mixture was subjected to pass through a short pad of silica gel. The filtrate obtained was evaporated under reduced pressure and subjected to flash column chromatography using silica gel. The titled compound F362 (0.25 g), which was eluted out after F363, was obtained in 36% yield and F363 (0.19 g) was obtained in 19% yield.

For F362: ¹H NMR (400 MHz, CHLOROFORM-d) δ 7.04-7.22 (m, 3H), 6.77 (t, J=9.29 Hz, 1H), 6.55 (dt, J=5.14, 9.17 Hz, 1H), 6.50 (br. s., 1H), 6.17 (br. s., 1H), 4.44 (br. s., 1H), 4.34 (d, J=5.87 Hz, 2H); ¹³C NMR (101 MHz, CHLOROFORM-d) δ 162.8, 162.6, 152.4, 151.9, 150.8, 150.1, 146.8, 135.6, 135.5, 133.2, 133.1, 122.9, 122.8, 122.8, 122.8, 117.6, 117.5, 116.0, 115.8, 113.5, 113.4, 113.4, 113.4, 111.5, 111.4, 111.3, 111.2, 46.9; LRMS (ESI) m/z 299 (M⁺+H, 100), 321 (M⁺+Na, 20); HRMS (ESI) calcd for C₁₄H₁₁N₂OF₄ (M⁺+H) 299.0808, found 299.0794;

For F363: ¹H NMR (400 MHz, CHLOROFORM-d) δ 6.94-7.15 (m, 7H), 6.88 (dd, J=3.18, 9.05 Hz, 1H), 6.68-6.78 (m, 1H), 6.39 (br. s., 1H), 4.15 (s, 4H); ¹³C NMR (101 MHz, CHLOROFORM-d) δ 163.0, 156.2, 154.9, 153.6, 152.5, 151.6, 151.5, 150.9, 150.7, 149.2, 149.0, 148.4, 148.3, 134.6, 134.6, 134.6, 134.5, 124.8, 124.5, 124.2, 124.2, 124.1, 124.1, 117.7, 117.6, 117.3, 117.1, 117.0, 116.9, 116.7, 114.7, 114.5, 114.3, 111.4, 111.4, 111.2, 111.1, 55.6; LRMS (ESI) m/z 425 (M⁺+H, 100), 447 (M⁺+Na, 28); HRMS (ESI) calcd for C₂₁H₁₅N₂OF₆ (M⁺+H) 425.1089, found 425.1082.

### Example 22: 3-((2′,4′-difluorobenzyl)amino)-2,6-difluorobenzamide (F364) and 3-(bis(2′,4′-difluorobenzyl)amino)-2,6-difluorobenzamide (F365)

These two compounds F364 (0.29 g, 42%) and F365 (0.21 g, 21%) were prepared from 2,6-difluoro-3-aminobenzamide (0.40 g, 2.3 mmol), 2,4-difluorobenzyl bromide (0.60 g, 2.9 mmol), ACN (20 mL) and K₂CO₃ (0.42 g, 3.0 mmol) according to the preparation procedure of F362 and F363 described above.

For F364: ¹H NMR (400 MHz, CHLOROFORM-d) δ 7.25-7.36 (m, 1H), 6.74-6.90 (m, 3H), 6.64 (dt, J=5.38, 9.05 Hz, 2H), 6.20 (br. s., 1H), 4.38 (s, 3H); ¹³C NMR (101 MHz, CHLOROFORM-d) δ 163.7, 163.5, 163.0, 162.6, 162.1, 161.9, 161.2, 161.1, 159.5, 152.4, 150.1, 150.0, 149.3, 133.1, 133.1, 133.0, 133.0, 130.0, 130.0, 129.9, 129.9, 121.3, 121.3, 121.2, 121.1, 113.5, 113.4, 113.4, 113.3, 112.9, 112.7, 111.6, 111.5, 111.5, 111.4, 111.4, 111.3, 111.3, 111.2, 104.3, 104.0, 103.8, 41.1; LRMS (ESI) m/z 299 (M⁺+H, 100), 321 (M⁺+Na, 20); HRMS (ESI) calcd for C₁₄H₁₁N₂OF₄ (M⁺+H) 299.0808, found 299.0793.

For F365: ¹HNMR (400 MHz, CHLOROFORM-d) δ 7.25-7.38 (m, 2H), 6.90 (dt, J=5.87, 9.05 Hz, 1H), 6.72-6.85 (m, 5H), 6.56 (br. s., 1H), 6.08 (br. s., 1H), 4.27 (s, 4H); ¹³C NMR (101 MHz, CHLOROFORM-d) δ 163.5, 163.4, 162.6, 162.2, 162.1, 161.1, 161.0, 159.8, 159.7, 134.5, 131.4, 131.3, 131.3, 131.2, 124.9, 120.3, 120.3, 120.2, 114.0, 111.5, 111.4, 111.3, 111.2, 111.2, 104.0, 103.7, 103.4, 49.3; LRMS (ESI) m/z 425 (M⁺+H, 100), 447 (M⁺+Na, 25); HRMS (ESI) calcd for C₂₁H₁₅N₂OF₆ (M⁺+H) 425.1089, found 425.1080.

### Example 23: 3-((2′,6′-difluorobenzyl)amino)-2,6-difluorobenzamide (F366) and 3-(bis(2′,6′-difluorobenzyl)amino)-2,6-difluorobenzamide (F367)

These two compounds F366 (0.29 g, 42%) and F367 (0.21 g, 21%) were prepared from 2,6-difluoro-3-aminobenzamide (0.40 g, 2.3 mmol), 2,6-difluorobenzyl bromide (0.60 g, 2.9 mmol), ACN (20 mL) and K₂CO₃ (0.42 g, 3.0 mmol) according to the preparation procedure of F362 and F363 described above.

For F366: ¹HNMR (400 MHz, CHLOROFORM-d) δ 7.22-7.31 (m, 1H), 6.78-6.95 (m, 4H), 6.38 (br. s., 1H), 6.12 (br. s., 1H), 4.45 (d, J=6.36 Hz, 2H), 4.34 (br. s., 1H); ¹³C NMR (101 MHz, CHLOROFORM-d) δ 162.8, 162.5, 160.4, 160.3, 152.6, 150.1, 149.6, 132.9, 129.8, 129.7, 129.6, 114.2, 114.0, 113.8, 111.7, 111.6, 111.5, 111.5, 111.5, 111.4, 111.3, 111.3, 35.8, 35.7; LRMS (ESI) m/z 299 (M⁺+H, 100), 321 (M⁺+Na, 30); HRMS (ESI) calcd for C₁₄H₁₁N₂OF₄ (M⁺+H) 299.0808, found 299.0800.

For F367: ¹H NMR (400 MHz, CHLOROFORM-d) δ 7.10-7.20 (m, 2H), 7.08 (br. s., 1H), 6.96 (dt, J=6.11, 8.93 Hz, 1H), 6.77 (t, J=7.82 Hz, 4H), 6.65 (t, J=9.05 Hz, 1H), 6.19 (br. s., 1H), 4.38 (s, 4H); ¹³C NMR (101 MHz, CHLOROFORM-d) δ 163.1, 163.0, 160.6, 160.5, 157.4, 157.3, 154.9, 154.8, 133.3, 133.3, 133.2, 133.2, 129.5, 129.4, 129.3, 127.8, 114.1, 113.9, 113.8, 113.7, 113.6, 113.4, 111.3, 111.2, 111.1, 111.0, 110.8, 110.8, 44.8; LRMS (ESI) m/z 425 (M⁺+H, 100), 447 (M⁺+Na, 40); HRMS (ESI) calcd for C₂₁H₁₅N₂OF₆ (M⁺+H) 425.1089, found 425.1082.

### Example 24: 2,6-difluoro-3-nonanamidobenzamide (F368)

To a well stirred solution of 2,6-difluoro-3-aminobenzamide (0.17 g, 1.0 mmol) in DCM (5 mL) and pyridine (5 mL) at 0° C. was added nonanoyl chloride (0.23 g, 1.3 mmol) dropwise. The reaction mixture was stirred for 4 hrs at 0° C. The reaction was then quenched by pouring the reaction mixture into a separating funnel containing 1 M HCl (50 mL) and extracted with DCM (20 mL×3). The combined organic layers were washed with NaHCO₃, dried over MgSO₄, filtered and evaporated under reduced pressure to give a crude reaction mixture, which was further subjected to flash column chromatography to afford the desired compound (0.11 g, 36%): ¹H NMR (400 MHz, Acetone-d₆) δ 8.95 (br. s., 1H), 8.16 (dt, J=6.36, 8.80 Hz, 1H), 7.51 (br. s., 1H), 7.19 (br. s., 1H), 7.02 (dt, J=1.71, 8.93 Hz, 1H), 2.47 (t, J=7.58 Hz, 2H), 1.70 (quin, J=7.34 Hz, 2H), 1.25-1.43 (m, 10H), 0.81-0.98 (m, 3H); ¹³C NMR (101 MHz, Acetone-d₆) δ 171.7, 161.2, 124.3, 123.6, 110.9, 110.7, 110.6, 36.3, 31.7, 25.3, 22.4, 19.1, 18.5, 13.4; LRMS (ESI) m/z 313 (M⁺+H, 100); HRMS (ESI) calcd for C₁₆H₂₂N₂O₂F₆ (M⁺+H) 313.1728, found 313.1716.

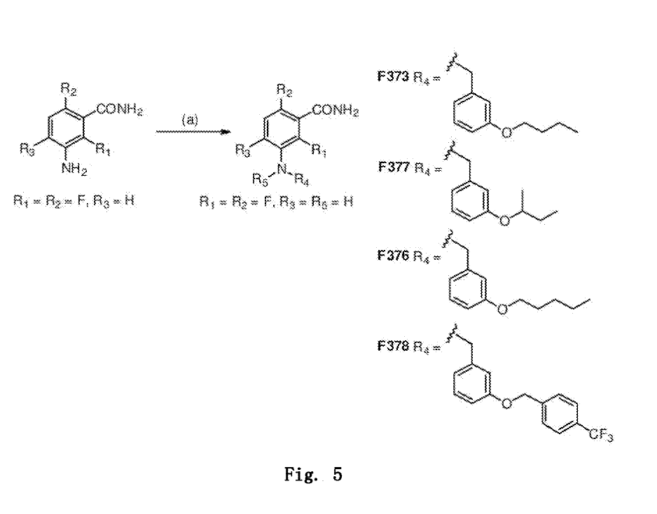

### The Fourth Preparation Method: Synthesis of Compounds F373, F376, F377 and F378 (See the Corresponding Compounds as Numbered in FIG. 5), Corresponding to the Following Examples 25-28

The chemical synthesis of the compounds F373, F376, F377 and F378 of the present invention were shown in FIG. 5. These compounds were prepared by reductive amination of 2,6-difluoro-3-aminobenzamide with corresponding benzaldehydes in good yield. The detailed experimental procedures were shown in FIG. 5. (a) various aldehydes, catalyst, pTsOH, MeOH, r.t., 2 hrs, then NaBH₃CN, 12 hrs.

### Example 25: 3-((3′-butoxybenzyl)amino)-2,6-difluorobenzamide (F373)

To a well stirred mixture of 2,6-difluoro-3-aminobenzamide (0.17 g, 1.0 mmol) and 3-butoxybenzaldehyde (0.17 g, 1.0 mmol) in MeOH (10 mL) at 0° C. was added p-toluenesulfonic acid monohydrate (0.02 g, 0.11 mmol) and the reaction mixture was stirred for 2 hrs. After that, excess sodium cyanoborohydride (0.63 g, 10.0 mmol) was added in portions to the reaction mixture. After the addition, the reaction mixture was stirred for further 12 hrs. The reaction was quenched by pouring the reaction mixture into a separating funnel containing 50 mL water and extracted with ethyl acetate (20 mL×3). The combined organic layers were dried over MgSO₄, filtered and evaporated under reduced pressure to give a crude product, which was subjected to flash column chromatography to afford the titled compound (0.15 g, 45%): ¹H NMR (400 MHz, CHLOROFORM-d) δ 7.26 (t, J=7.82 Hz, 1H), 6.86-6.97 (m, 2H), 6.73-6.86 (m, 2H), 6.63 (dt, J=5.38, 9.05 Hz, 1H), 6.56 (br. s., 1H), 6.16 (br. s., 1H), 4.27-4.40 (m, 3H), 3.96 (t, J=6.36 Hz, 2H), 1.71-1.81 (m, 2H), 1.44-1.57 (m, 2H), 0.99 (t, J=7.34 Hz, 3H); ¹³C NMR (101 MHz, CHLOROFORM-d) δ 163.1, 159.6, 152.2, 149.2, 146.7, 140.0, 133.7, 133.5, 129.8, 119.2, 113.5, 113.5, 113.4, 113.4, 111.4, 111.4, 111.2, 111.2, 67.7, 47.9, 31.3, 19.2, 13.9; LRMS (ESI) m/z 335 (M⁺+H, 60), 357 (M⁺+Na, 50); HRMS (ESI) calcd for C₁₈H₂₁N₂O₂F₂ (M⁺+H) 335.1571, found 335.1568.

### Example 26: 2,6-difluoro-3-((3′-(pentyloxy)benzyl)amino)benzamide (F376)

This compound (0.13 g, 38%) was prepared from 2,6-difluoro-3-aminobenzamide (0.17 g, 1.0 mmol), 3-(pentyloxy)benzaldehyde (0.19 g, 1.0 mmol), p-toluenesulfonic acid monohydrate (0.02 g, 0.11 mmol), MeOH (10 mL) and sodium cyanoborohydride (0.63 g, 10 mmol) according to the preparation procedure of F373 described above: ¹H NMR (400 MHz, Acetone-d₆) δ 7.37 (br. s., 1H), 7.24 (t, J=7.82 Hz, 1H), 7.10 (br. s., 1H), 6.93-7.01 (m, 2H), 6.72-6.85 (m, 2H), 6.65 (dt, J=5.38, 9.29 Hz, 1H), 5.54 (br. s., 1H), 4.42 (d, J=5.87 Hz, 2H), 3.92-4.02 (m, 2H), 1.71-1.82 (m, 2H), 1.34-1.49 (m, 4H), 0.87-0.97 (m, 3H); ¹³C NMR (101 MHz, Acetone-d₆) δ 159.6, 141.3, 129.4, 119.1, 113.3, 112.7, 112.2, 110.4, 67.5, 46.9, 22.2, 13.4; LRMS (ESI) m/z 349 (M⁺+H, 100), 371 (M⁺+Na, 50); HRMS (ESI) calcd for C₁₉H₂₃N₂O₂F₂ (M⁺+H) 349.1728, found 349.1739.

### Example 27: 3-((3′-(sec-butoxy)benzyl)amino)-2,6-difluorobenzamide (F377)

This compound (0.12 g, 36%) was prepared from 2,6-difluoro-3-aminobenzamide (0.17 g, 1.0 mmol), 3-(sec-butoxy)benzaldehyde (0.17 g, 1.0 mmol), p-toluenesulfonic acid monohydrate (0.02 g, 0.11 mmol), MeOH (10 mL) and sodium cyanoborohydride (0.63 g, 10 mmol) according to the preparation procedure of F373 described above: ¹H NMR (400 MHz, CHLOROFORM-d) δ 7.24 (t, J=8.07 Hz, 1H), 6.85-6.93 (m, 2H), 6.71-6.85 (m, 3H), 6.61 (dt, J=5.14, 9.17 Hz, 1H), 6.27 (br. s., 1H), 4.38 (br. s., 1H), 4.27-4.34 (m, 3H), 1.68-1.79 (m, 1H), 1.57-1.67 (m, 1H), 1.25-1.31 (m, 3H), 0.97 (t, J=7.58 Hz, 3H); ¹³C NMR (101 MHz, CHLOROFORM-d) δ 163.3, 158.7, 152.2, 149.8, 149.7, 149.2, 149.1, 146.7, 146.6, 140.1, 133.7, 133.6, 133.6, 133.5, 129.8, 119.1, 114.8, 114.6, 113.5, 113.4, 113.4, 113.3, 112.9, 112.8, 112.6, 111.4, 111.4, 111.2, 111.1, 75.0, 47.9, 29.2, 19.2, 9.8; LRMS (ESI) m/z 335 (M⁻¹+H, 40), 357 (M⁺+Na, 40); HRMS (ESI) calcd for C₁₈H₂₁N₂O₂F₂ (M⁺+H) 335.1571, found 335.1580.

### Example 28

### 2,6-difluoro-3-((3′-((4″-(trifluoromethyl)benzyl)oxy)benzyl)amino)benzamide (F378)

This compound (0.16 g, 37%) was prepared from 2,6-difluoro-3-aminobenzamide (0.17 g, 1.0 mmol), 3-((4-(trifluoromethyl)benzyl) oxy)benzaldehyde (0.28 g, 1.0 mmol), p-toluenesulfonic acid monohydrate (0.02 g, 0.11 mmol), MeOH (10 mL) and sodium cyanoborohydride (0.63 g, 10 mmol) according to the preparation procedure of F373 described above: ¹H NMR (400 MHz, CHLOROFORM-d) δ 7.66 (d, J=8.31 Hz, 2H), 7.56 (d, J=7.82 Hz, 2H), 7.29 (d, J=6.85 Hz, 1H), 6.94-7.03 (m, 2H), 6.88-6.93 (m, 1H), 6.72-6.83 (m, 1H), 6.62 (td, J=4.59, 8.93 Hz, 1H), 6.25 (br. s., 1H), 6.09 (br. s., 1H), 5.13 (s, 2H), 4.36 (s, 3H); ¹³C NMR (101 MHz, CHLOROFORM-d) δ 158.9, 141.0, 140.3, 130.0, 127.4, 125.6, 125.5, 125.5, 120.0, 113.7, 113.6, 111.4, 69.1, 47.8; LRMS (ESI) m/z 437 (M⁺+H, 56), 459 (M⁺+Na, 50); HRMS (ESI) calcd for C₂₂H₁₈N₂O₂F₅ (M⁺+H) 437.1288, found 437.1292.

The derivatives of the present invention were subjected to the following biological tests.

(A) Bacterial Strains and Cell Lines

The bacterial strains used in microbial susceptibility test were E. coli, S. aureus 29213 and B. subtilis 168. E. coli and S. aureus 29213 were obtained from the laboratory of the Hong Kong Polytechnic University. B. subtilis 168 was purchased from American Type Culture Collection (USA). The mammalian cell lines L929 (mouse peritoneal fibroblast), LCC6 (a wild type breast cancer cell line), LCC6MDR (P-gp over-expressed breast cancer cell line) were stocks in our department. Nutrient Agar, tryptone and yeast extract were purchased from Oxoid Limited (Nepean, Ontario, Canada). Luria-Bertani (LB) medium was prepared from 2 g tryptone, 1 g yeast extract, and 2 g sodium chloride in 200 ml deionized water and sterilized. Müller-Hinton broth (MHB), cation-adjusted Müller-Hinton broth (MHB) and trypticase soy broth (TSB) for microbial susceptibility test were purchased from Becton, Dickinson and Company (New Jersey, USA). A microplate reader (Bio-Rad Laboratories Model 680) is used to measure bacterial growth and cytotoxicity. An Olympus FSX100® Bio Imaging Navigator Microscope was used to observe the morphology of B. subtilis cell.

(B) Antibacterial Susceptibility Test

The minimum inhibitory concentration (MIC) of each compound was determined using microbroth dilution method according to the National Committee for Clinical Laboratory Standards (CLSI) guidelines. All Compounds were dissolved in DMSO and diluted in a serial two-fold manner. A single colony of bacterial strain on a TSB agar plate was inoculated in 5 mL MHB or CA-MHB. The bacterial cells were incubated at 37° C. until the optimal density (OD₆₀₀) of the growing cells reached 1.0. The cells were then diluted to a final concentration of approximately 5×10⁵ CFU/mL in a serial two-fold manner in a 96-well microtiter plate (Iwaki, Japan). Compound 3 and PC190723 were used as positive controls. After 18 hours of incubation at 37° C., the OD₆₀₀ values were measured by a microplate absorbance reader to calculate the percentage inhibition of bacterial growth with respect to control. The compound is defined to be active if the MIC value is equal to or greater than 90%, which means that the compound causes more than 90% inhibition of bacterial growth. Table 1 below summarizes the MIC of all synthesized compounds.

TABLE 1
MIC values of each compound against different bacterial strains.

| | | Strain | | |
|---|---|---|---|---|
| Compound | | BS 168 (μM) | E. coli (μM) | S.A 29213 (μM) |
| PC190723 | | 0.5 | >2 | 0.5 |
| Formula 3 | | 0.25 | >100 | 6.25 |
| compounds | F411 | 6.25 | >100 | >100 |
| obtained by | F412 | 25 | >100 | >100 |
| the 1^{st} | F413 | >100 | >100 | >100 |
| preparation | F414 | >100 | >100 | >100 |
| method | F342 | 25 | >100 | >100 |
| | F345 | 100 | >100 | >100 |
| compounds | F391 | 50 | >100 | 50 |
| obtained by | F333 | 6.25 | >100 | 50 |
| the 2^{nd} | F369 | 50 | >100 | >100 |
| preparation | F370 | 6.25 | >100 | 25 |
| method | F332 | 3.13 | >100 | 12.5 |
| | F371 | 6.25 | >100 | >100 |
| | F350 | >100 | >100 | >100 |
| | F334 | >100 | >100 | >100 |
| | F409 | 25 | >100 | 12.5 |
| | F410 | 25 | >100 | 50 |
| | F361 | 1.57 | >100 | 3.13 |
| compounds | F368 | >100 | >100 | >100 |
| obtained by | F357 | >100 | >100 | >100 |
| the 3^{rd} | F358 | >100 | >100 | >100 |
| preparation | F362 | >100 | >100 | >100 |
| method | F363 | >100 | >100 | 50 |
| | F364 | >100 | >100 | >100 |
| | F365 | <50 | >100 | >100 |
| | F366 | >100 | >100 | >100 |
| | F367 | >100 | >100 | >100 |
| compounds | F373 | 25 | >100 | 50 |
| obtained by | F377 | >100 | >100 | >100 |
| the 4^{th} | F376 | 12.5 | >100 | 50 |
| preparation | F378 | >100 | >100 | >100 |
| method | | | | |

(C) Cytotoxicity Test

The cell lines used in cytotoxicity test were stocks in our department. In this assay, a mouse fibroblast cell line (L929), a wild type breast cancer cell line (LCC6) and a P-gp-over-expressed breast cancer cell line (LCC6MDR) were mixed with compounds of various concentrations and made up to a final volume of 100 μL in 96-well plates. The cells were then incubated at 37° C. for 3 days. The half-maximal inhibitions (IC₅₀) of the compounds were determined using the CellTiter 96 AQueous One Solution cell Proliferation Assay (Promega). 3-(4,5-Dimethylthiazol-2-yl)-5-(3-carboxy-methoxyphenyl)-2-(4-sulfophenyl)-2H-tetr azolium (2 mg/mL, MTS, Promega) and phenzaine methosulfate (0.92 mg/mL PMS, Sigma-Aldrich) were mixed in a ratio of 20:1. The MTS/PMS mixture (10 μL) was added into each well, and the plate was incubated at 37° C. for 2 hours. The absorbance at 490 nm was then measured with a microplate absorbance reader (Bio-Rad). The IC₅₀ values were calculated using GraphPad (Prism 4.0). The selective index of each compound was calculated from the ratio between the toxicity level of L929 and the MIC value of S. aureus 29213. A compound is defined as a potential antibiotic if the selective index is greater than 10. Two promising compounds F332 and F361 were chosen to test on three mammalian cell lines. The results were shown in Table 2.

TABLE 2
IC₅₀ values of F332 and F361 against different mammalian cell lines.

| | Mammalian Cell Line | | |
|---|---|---|---|
| | LCC6 (IC₅₀, | | |
| Compounds | μM) | LCC6MDR (IC₅₀, μM) | L929 (IC₅₀, μM) |
| F332 | 78 | >100 | >100 |
| F361 | 60 | 52 | 60 |

(D) Effects on the Morphology of B. subtilis Cells

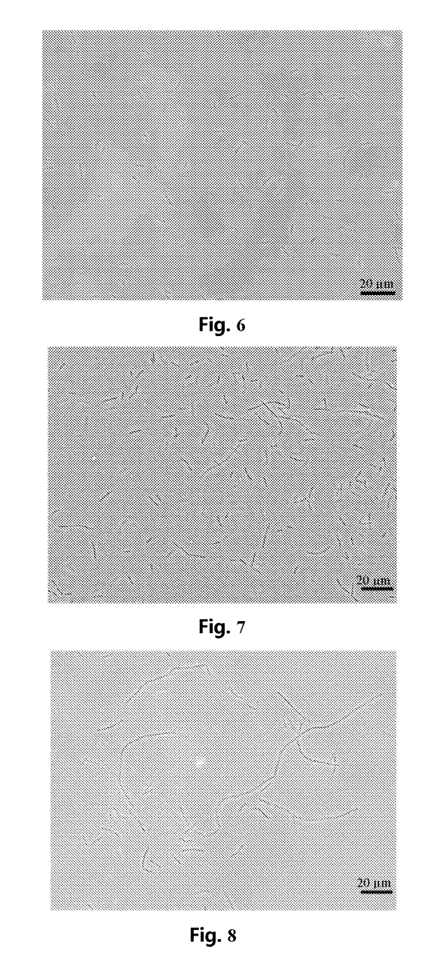

The two most promising compounds, F332 and F361, were tested for their effects on the cell morphology of B. subtilis 168 strain. The log-phase culture of B. subtilis was tested with the two compounds with a final concentration of half of MIC value. A 1% DMSO solution was used as the control. The results indicated that both compounds had the ability to induce filamentation on B. subtilis cells. The average cell length of untreated B. subtilis cell was 3.5±0.7 μm (FIG. 6). F332 induced a three-fold lengthening in the cell length of B. subtilis (FIG. 7). F332 extended the average cell length of B. subtilis from 3.5±0.7 μm to 11.7±4.5 μm. On the other hand, F361 induced a six-fold lengthening of cell length in B. subtilis, from 3.5±0.7 μm to 21.1±1.8 μm (FIG. 8). F332 and F361 were found to cause filamentation of B. subtilis 168 cells at sub-lethal concentrations. The results showed that both compounds may inhibit bacterial proliferation by blocking cytoplasmic cell division protein.

(E) Fluorescence Microscopy

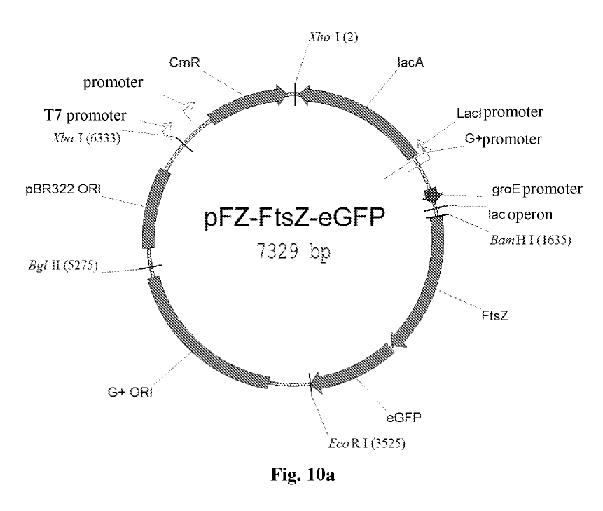

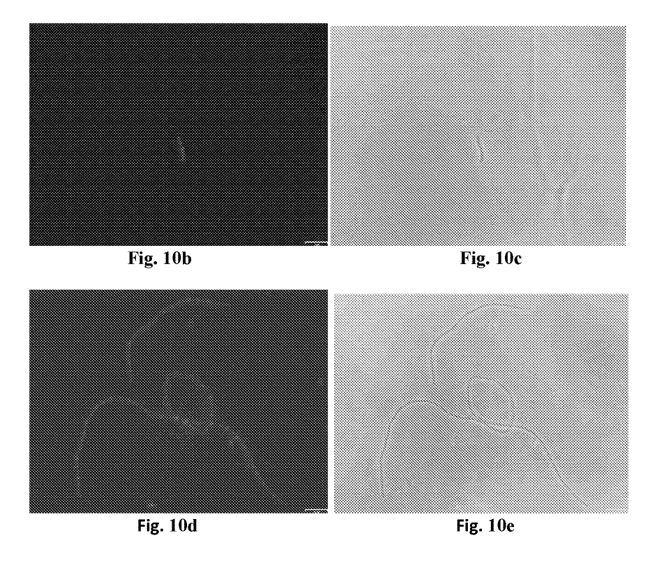

The localization of the filamentation in B. subtilis that induced by F332 was performed by fluorescence microscopy using the cell division protein FtsZ identified by a green fluorescent protein. The expression plasmid of the FtsZ-eGFP fusion protein was constructed as follows (the structure of the plasmid was shown in FIG. 10a).

A pair of primers were designed according to the DNA sequence of the plasmid pGK12 of Gram-positive bacteria (G+)

Sequence 1
5′-TTTCCCTCTAGACCGGCAATAGTTACCCTTATT-3′
Sequence 2
5′-TGACAGAGATCTCATTTTGTTTATTGCAATTG-3′

to amplify a PCR fragment, with about 4,000 base pairs in length, containing chloramphenicol resistance gene, G+ replication origin and most of the pGK12 sequence.

In order to introduce the origin of replication of Gram-negative bacteria (G-) and enable the chloramphenicol resistance gene to be efficiently expressed in E. coli, a pair of primers were designed according to the DNA sequence of the G-plasmid pRSET-A (Invitrogen)

Sequence 3
5′-AAAATGAGATCTCTGTCAGACCAAGTTTACTC-3′
Sequence 4
5′-TGCCGGTCTAGAGGGAAACCGTTGTGGTCT-3′

to amplify a PCR fragment, with about 1,000 base pairs in length, containing the origin of replication of pBR322 and the T7 promoter. Two PCR fragments digested with restriction endonucleases XbaI and BgIII were ligated and transformed into E. coli BL21 (DE3) and screened on chloramphenicol plates to obtain the plasmid pFZ1.0.

By introducing the lactose operon mechanism, the lethal effect of the host caused by overexpression of the FtsZ gene of B. subtilis can be effectively controlled. A pair of primers were designed according to the DNA sequence of pFZ1.0

Sequence 5
5′-AAGTAAGAATTCTGCTAGCAGAAGCTTCG-3′
Sequence 6
5′-GGGGCACTCGAGGGGTTAGTGACATTAGAAAAC-3′

to amplify a PCR fragment of about 3,800 base pairs, removing the excess part of the pGK12.

A pair of primers were designed according to the DNA sequence of pMAL-c2 (NEB)

Sequence 7
5′-ATCCTACTCGAGTGCCCCGTTAGTTGAAGAAG-3′
Sequence 8
5′-TAGCATGAATTCAAACGATCCCGCGAAATTAATAC-3′

to amplify a PCR fragment, with about 1,200 base pairs, containing the lacA gene and its lad promoter. Two PCR fragments digested with restriction endonucleases XhoI and EcoRI were ligated and transformed into E. coli BL21 (DE3) and screened on chloramphenicol plates to obtain the plasmid pFZ1.1.

A pair of primers were designed according to the DNA sequence of pFZ1.1

Sequence 9
5′-AAGTAAGAATTCTGCTAGCAGAAGCTTCG-3′
Sequence 10
5′-AGATCTGGATCCAAACGATCCCGCGAAATTAATAC-3′

to amplify a PCR fragment, with about 5,000 base pairs, and the fragments were digested with restriction endonucleases EcoRI and BamHI.

A pair of primers were then designed according to the DNA sequence of G+ vector pHT43 (MoBiTec)

Sequence 11
5′-GGATCCAGATCTGAATATTTCAGCTTGGTTTTCC-3′
Sequence 12
5′-AGCTAAGAATTCGCTACGATGGATCCTTCCTCCTTTAATTGG-3′

to amplify a PCR fragment, with about 450 base pairs, containing the groE promoter and the lacO sequence. Two fragments digested with restriction endonucleases EcoRI and BgIII were ligated and transformed into E. coli BL21 (DE3) and screened on chloramphenicol plates to obtain the plasmid pFZ2.0.

A pair of primers were designed according to the FtsZ gene sequence of B. subtilis 168

Sequence 13
5′-GAGGAAGGATCCATGTTGGAGTTCGAAACAAAC-3′
Sequence 14
5′-ACTGCCCGAACCGCTTCCGCCGCGTTTATTACGG-3′

to amplify a PCR fragment, with about 1,200 base pairs.

In addition, a pair of primers were designed according to the DNA sequence of pEGFP

Sequence 15
5′-GGAAGCGGTTCGGGCAGTATGGTGAGCAAGGGCGA-3′
Sequence 16
5′-CTAGCAGAATTCTTACTTGTACAGCTCGTCC-3′

to amplify the eGFP gene fragment, with about 700 base pairs. Two fragments were ligated by overlap extension PCR method and then digested with endonucleases EcoRI and BamHI. They were ligated into the corresponding cleavage site of pFZ2.0 and transformed into E. coli BL21 (DE3) and screened on chloramphenicol plates to obtain the plasmid pFZ-FtsZ-eGFP. The plasmid extracted was transformed into B. subtilis 168 and grown in LB medium containing chloramphenicol. The expression of FtsZ-eGFP gene was observed by adding different concentrations of lactose or IPTG

The B. subtilis shown in FIGS. 10b and 10c was cultured in the absence of F332. Strong green fluorescence appeared in the middle part of the B. subtilis cells in FIG. 10b under fluorescence microscopy, indicating that the expression plasmid of the FtsZ-eGFP fusion protein was localized and integrated into the middle of the cell to form a Z-ring in cell division.

The B. subtilis shown in FIGS. 10d and 10e was cultured in the presence of F332. Green fluorescence appeared in multiple parts of B. subtilis cells in FIG. 10d under fluorescence microscopy and the cells were grown, reflecting that the localization of the expression plasmid of the FtsZ-eGFP fusion protein in the cells was affected by F332. Thus, the expression of the protein was not only at the position where cell division was performed.

(F) Expression and Purification of S. aureus FtsZ Protein

E. coli BL21 cells carrying S. aureus ftsz gene were obtained from a stock in our laboratory. The E. coli strain from the stock was incubated in 5 mL LB medium in the presence of 100 μg/mL ampicillin at 37° C. for 14-16 hours. The bacterial culture (2 mL) from overnight culture was transferred to 200 mL of 2×TY medium containing 100 μg/mL ampicillin and allowed to grow at 37° C. until the optical absorbance (OD₆₀₀) reached 0.8. Protein expression was induced by the addition of 0.4 mM isopropyl-β-D-1-thiogalactopyranoside (IPTG) for another 4 hours. The bacterial cells were collected by centrifugation at 9000 rpm at 4° C. for 20 minutes. Cell pellets were then re-suspended in 20 mL starting buffer (0.02 M sodium phosphate, 0.5 M sodium chloride, pH 7.4). The cells were homogenized by sonication operated at 50% amplitude with 30 seconds pulse-on and 30 seconds pulse-off for 5 cycles. The insoluble cell debris was removed by centrifugation at 13,000 rpm at 4° C. for 1.5 hours. The supernatant was collected and filtered by a sterile 0.22 μm filter.

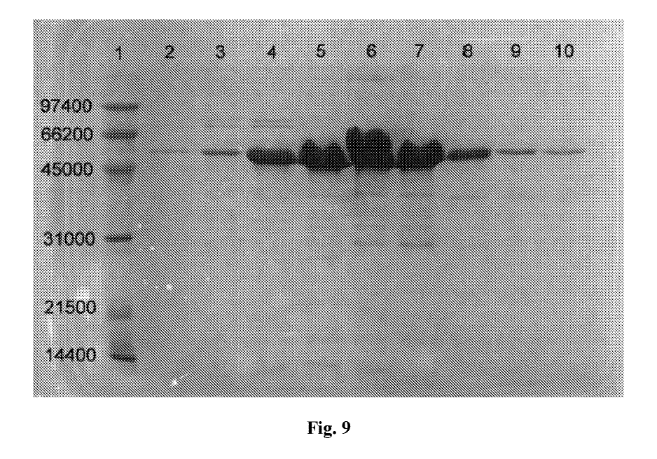

The FtsZ protein in supernatant was purified by Fast Protein Liquid Chromatography (FPLC). A 5 mL HiTrap chelating column activated by nickel (II) sulphate solution was used in FPLC in order to capture S. aureus FtsZ proteins. The activated column was then equilibrated by starting buffer. The soluble protein solution containing the target protein was passed through the column with starting buffer. The target protein (Peak A) was then eluted with elution buffer (0.02 M sodium phosphate, 0.5 M sodium chloride, 0.5 M imidazole, pH 7.4). The fractions were collected and analyzed by SDS-PAGE (FIG. 9), followed by dialysis against 50 mM MOPS buffer and stored at −80° C.

(G) Light Scattering Assay

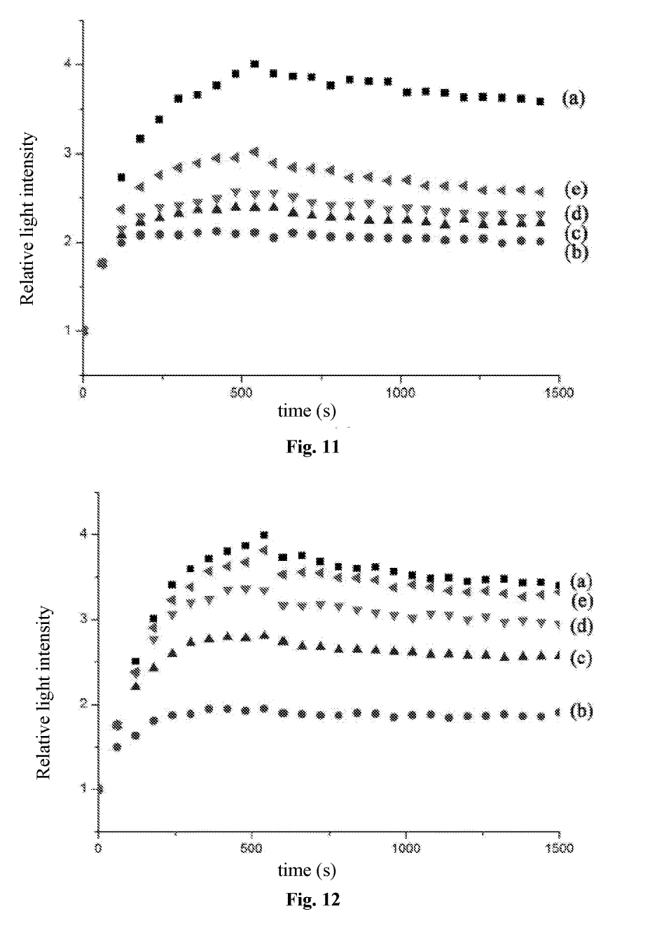

Light scattering assay was used to determine the degree of FtsZ polymerization by measuring the 90° light scattering signal using a fluorescence spectrometer (Perkin-Elmer, USA). The excitation and emission wavelengths were set to 600 nm with a slit width of 5 nm. S. aureus FtsZ (12 μM) was incubated with serial dilutions of the compounds in 50 mM MOPS buffer (pH 6.8) at 25° C. for 10 minutes. The control contained 1% DMSO only. KCl (final concentration: 50 mM) and MgCl₂ (final concentration: 10 mM) were then added to the mixture. The mixture was incubated at 37° C. for 8 minutes in order to establish a baseline. A final concentration of 1 mM GTP was added and the light scattering signal was measured for 1,500 seconds. The relative light scattering signals in experimental set-up were calculated with reference to the light scattering signal in control experiment. Three independent experiments were carried out for each compound concentration. Two promising compounds F332 and F361 were chosen for testing and their results were shown in FIGS. 11 and 12 respectively. Both compounds can inhibit the light scattering of FtsZ assembly. The decreased light scattering signal indicated that both compounds can reduce the polymer mass of FtsZ proto-filament in a dose-dependent manner.

(H) GTPase Activity Assay

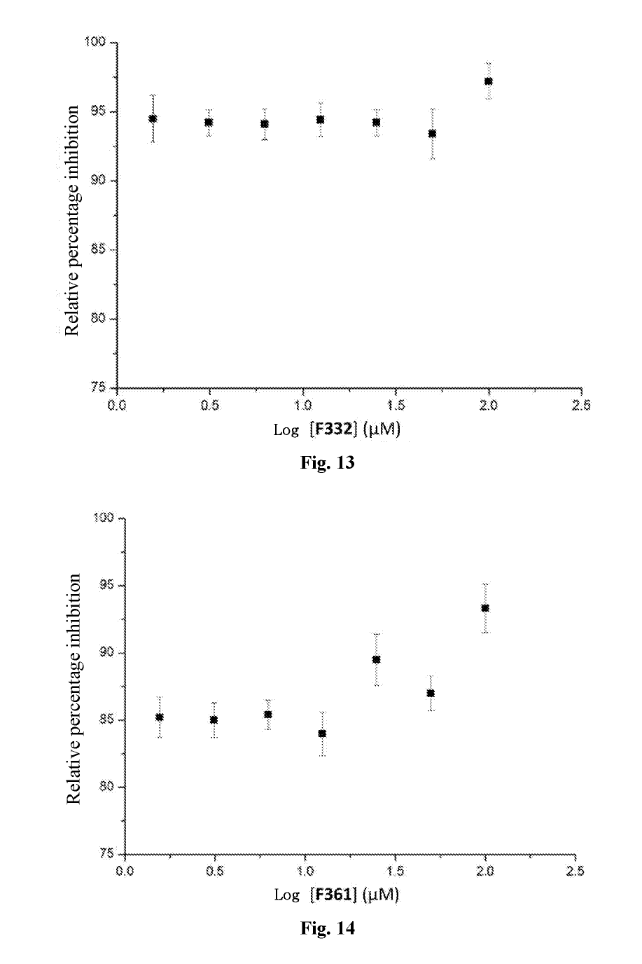

The GTPase activity of S. aureus FtsZ was determined in a 96-well microplate using a CytoPhos™ phosphate assay Biochem Kit™ (Cytoskeleton, USA) which measured the concentration of inorganic phosphate released during the hydrolysis of GTP. Recombinant S. aureus FtsZ (7.5 μM) was incubated with serial dilutions of the compounds in 50 mM MOPS buffer (pH 6.5) at 25° C. for 10 minutes. The control contained 1% DMSO only. KCl (final concentration: 200 mM) and MgCl₂ (final concentration: 5 mM) were then added to the mixture. The mixture was incubated with GTP (final concentration: 500 μM) at 37° C. for 30 minutes. The insoluble polymer was removed by centrifugation at 13,500 rpm at room temperature for 1 minute. 80 μL supernatant was transferred to a well of a 96-well microplate and incubated with 120 μL of Cytophos reagent at room temperature for 10 minutes. The inorganic phosphate in the mixture was determined by measuring the absorbance at 650 nm in a 96-well microplate reader (BioRad 680). The results from the samples were reported with reference to the control. Three independent experiments were carried out for each compound concentration. Two promising compounds F332 and F361 were chosen for testing and their results were shown in FIGS. 13 and 14 respectively. As shown in the FIGS. 13 and 14, no significant change of the GTPase activity of S. aureus FtsZ was observed upon addition of both compounds. Based on the light scattering results and GTPase activity measurement, it seems very likely that both compounds inhibit the polymerization of FtsZ without affecting its GTPase activity.

Transmission Electron Microscopy (TEM)

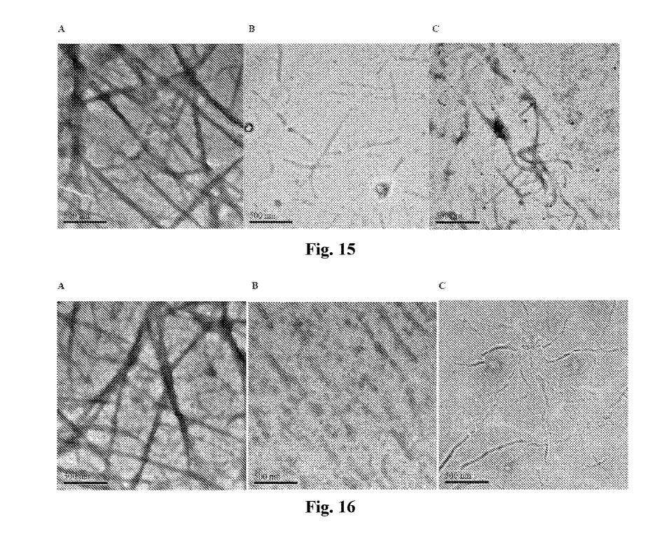

TEM provides a direct observation of FtsZ polymer morphology. Two promising compounds F332 and F361 were chosen for testing. S. aureus FtsZ (12 NM) was incubated with the compounds at different concentrations in 50 mM MOPS buffer (pH 6.5) at 25° C. for 10 minutes. The control contained 1% DMSO only. KCl (final concentration: 50 mM) and MgCl₂ (final concentration: 5 mM) were then added to the mixture. The mixture was incubated with GTP (final concentration: 1 mM) at 37° C. for 15 minutes. 10 μL reaction mixtures were applied on a glow-discharged Formvar carbon-coated copper grid (400 meshes) at 25° C. for 10 minutes. 10 μL of 0.5% phosphotungistic acid (PTA) was applied on the copper grid in order to negatively stain the proteins. The copper grid was allowed to air dry overnight. The FtsZ polymer morphology was observed with a transmission electron microscope (JEOL model JEM 2010) operated at 200 kV and equipped with a Gutan MSC 794 CCD camera. The electron micrographs of F332 and F361 were shown in FIGS. 15 and 16 respectively. An extensive network of thick bundles formed by FtsZ protofilaments was observed in the absence of inhibitor. Comparing with protofilaments obtained in the presence of inhibitor and relatively short and straight single strand FtsZ protofilaments obtained when FtsZ was polymerized in the presence of 100 μM of F332 or F361. This phenomenon coincides with the light scattering assay, where both compounds were shown to inhibit FtsZ polymerization in a dose-dependent manner.

Determination of the Fractional Inhibitory Concentration Index (FICI)

The bacterial strains used in this study were clinically isolated MRSA strains USA300#757 and #2960 which were obtained from our laboratory. Determination of the Fractional Inhibitory Concentration Index (FICI) was adopted by the Müller-Hinton broth (MHB) dilution method as previously described. Twelve serial two-fold dilutions of PC190723, F332 and F361 were prepared in the 96-well microplate with concentration ranging from 512 to 0.25 μg/mL. A series of two-fold dilutions of ampicillin, cloxacillin and cefuroxime were also prepared following the same procedure. Lastly, the compounds and the β-lactams were mixed at a 1:1 ratio and subjected to two-fold dilution as described previously. Each microplate well contained 100 μL of MHB supplemented with compounds alone, β-lactams alone or the mixture of compounds and β-lactams. Overnight cultures of S. aureus in the Müller-Hinton broth was diluted to OD₅₉₅ of 0.08-0.1 in saline and added in each of the microplate well. The plates were incubated at 37° C. for 20 h. All tests were performed in duplicate and the inhibition of bacterial growth was determined by visualization. The FICI of compounds and β-lactams were calculated as followed: FICI=(MIC of combination of compound and β-lactam/MIC of compound alone)+(MIC of combination of compound and β-lactam/MIC of β-lactam alone). The results were summarized in Table 3.

TABLE 3
FICI values of PC190723, F332 and F361 against two MRSA strains
(USA300#757; USA300#2960).

| | Antibiotics | | | | |
|---|---|---|---|---|---|
| | | | | Clox- | Cefur- |
| compound | Cefotaxime | Meropenem | Ampicillin | acillin | oxime |

USA300#757

| | | | | | |
|---|---|---|---|---|---|
| PC190723 | 1.02 | 0.31 | n.d. | n.d. | n.d. |
| F332 | 0.13 | 1.06 | 0.52 | 1.02 | 0.09 |
| F361 | 0.38 | 1.13 | n.d. | n.d. | n.d. |

USA300#2960

| | | | | | |
|---|---|---|---|---|---|
| PC190723 | 1.03 | 0.63 | n.d. | n.d. | n.d. |
| F332 | 0.38 | 0.28 | 0.28 | 1.02 | 0.38 |
| F361 | 1.00 | 0.28 | n.d. | n.d. | n.d. |

In order to further demonstrate that it can be applied to different strains, F332 was applied to the strains in Table 4 in combination with five antibiotics respectively, and the different FICI values were shown in Table 4.

TABLE 4
FICI values of F332 combined with the following antibiotics against
different strains.

| | FICI values of F332 combined with the following | | | | |
|---|---|---|---|---|---|
| | antibiotics against different strains | | | | |
| MRSA | | | | Cefur- | |
| strain | Amoxicillin | Meticillin | Cloxacillin | oxime | Meropenem |
| 2490 | 0.56 | 0.25 | 0.56 | 0.19 | 2.25 |
| 254 | 0.56 | 0.25 | 0.19 | 0.25 | 1.50 |
| 231 | 0.52 | 0.50 | 0.28 | 0.50 | 0.63 |
| 417 | 0.27 | 0.06 | 0.09 | 0.06 | 0.19 |
| 1344 | 1.13 | 0.27 | 0.13 | 0.27 | 2.00 |
| 2901 | 0.06 | 0.01 | 0.25 | 0.13 | 4.01 |
| 3589 | 1.06 | 0.52 | 0.31 | 0.25 | 1.50 |
| 256 | 0.08 | 0.04 | 0.13 | 0.01 | 4.01 |
| 3332 | 0.63 | 0.28 | 0.38 | 0.13 | 2.25 |
| 3411 | 2.02 | 1.02 | 0.53 | 1.00 | 3.00 |
| 1136 | 1.03 | 0.31 | 0.38 | 0.13 | 4.50 |
| 2895 | 1.06 | 0.31 | 0.38 | 0.31 | 2.13 |
| 2516 | 0.38 | 0.19 | 0.19 | 0.07 | 0.38 |
| 3267 | 0.25 | 0.07 | 0.25 | 0.01 | 4.01 |
| 757 | 0.27 | 0.09 | 0.13 | 0.08 | 1.06 |
| #1749 | 1.03 | 1.06 | 1.25 | 1.13 | 10.0 |
| 1402 | 1.02 | 0.56 | 0.75 | 0.63 | 5.00 |
| 2919 | 0.51 | 0.38 | 0.31 | 0.25 | 4.25 |
| 2728 | 0.08 | 0.16 | 1.01 | 0.14 | 2.03 |
| #1717 | 1.06 | 1.13 | 2.50 | 1.13 | 3.00 |
| 2224 | 0.19 | 0.13 | 0.25 | 0.25 | 4.01 |
| 358 | 0.27 | 0.25 | 1.06 | 1.13 | 129.0 |
| 3580 | 0.28 | 0.19 | 0.53 | 0.06 | 1.25 |
| 774 | 0.03 | 0.26 | 0.50 | 0.01 | 0.13 |
| 200 | 0.02 | 0.25 | 1.00 | 1.00 | 8.01 |
| 2973 | 0.08 | 0.28 | 0.07 | 0.53 | 4.03 |
| 85 | 0.04 | 0.50 | 1.00 | 0.25 | 4.01 |
| 1449 | 0.38 | 0.75 | 0.63 | 1.50 | 33.0 |

#ATCC strains

Study on Pharmacokinetic Parameters and its Application in Drug Preparation

Compound F332 was further subjected to in vivo pharmacokinetic study. The UPLC-MS/MS system consists of an Acquity Waters UPLC interfaced with triple quadrupole mass spectrometer (Micromass model Quattro Ultima) equipped with an electrospray ionization source in positive mode. Chromatographic separation was done on ACQUITY UPLC BEH C18 1.7 μM (2.1×50 mm) column. The mobile phase consists of methanol+0.1% formic acid (solvent B) and Milli-Q water+0.1% formic acid (solvent A). Multiple reaction monitoring (MRM) was set monitoring the transitions for F332 [M+H]⁺ at 299>142 m/z. The collision energy, cone voltage, source temperature, desolvation temperature and capillary voltage are 25, 30, 150° C., 350° C. and 3 Kv, respectively. The cone gas and desolvation gas was 150 L/Hr and 600 L/Hr, respectively.

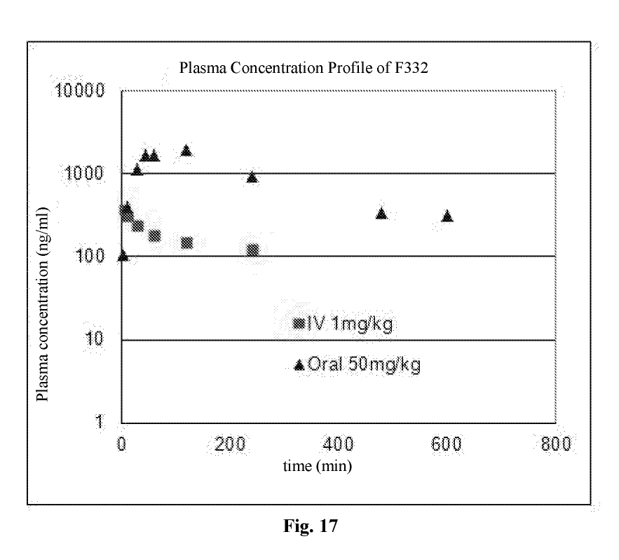

Male Sprague-Dawley (SD) rats (body weight 250-280 g) were obtained from Centralised Animal Facilities, The Hong Kong Polytechnic University. Animals were kept in a temperature and humidity controlled environment with 12 hour light-dark cycle with standard diet and water. Animal experiment protocol was approved by the Animal Subjects Ethics Sub-Committee of The Hong Kong Polytechnic University. Right jugular vein cannulation was done one day in advance of the experiment. Animal were fasted overnight and had free access to water throughout the experiment. F332 was freshly prepared in 5% CremophorEL, 5% ethanol, 90% saline at a concentration of 2 mg/ml. F332 was prepared on the day of use and used for animal study within half an hour. In the current study, F332 was administered through passive oral feeding (oral) and intravenous (IV) injection. Blood samples (approx. 500 μL) were collected in heparinzied tubes (20 units of heparin salt/tube) via jugular vein at 5, 10, 30, 45, 60, 120, 240 and 420 minutes post administration for IV study. As for oral study, plasma samples were collected at 2, 10, 30, 45, 60, 120, 240, 480 and 600 minutes. Blood plasma samples were collected by centrifuged at 16,100 G for 10 minutes. For all blood plasma samples, 3 fold volume of methanol was added for protein precipitation. Supernatant was filtered by a syringe filter before UPLC-MS/MS analysis. Pharmacokinetic parameters in plasma were determined by non-compartmental pharmacokinetics data analysis using Pharmacokinetics Solutions 2.0 software (Summit Research Services, Montrose, Colo., USA). The pharmacokinetic parameters determined include maximal plasma concentration (Cmax), time to reach maximal plasma concentration (Tmax), mean residence time (MRT), absorption/distribution half life (t1/2α), elimination half life (t1/2β), volume of distribution (Vd), systemic clearance (CL) and the area under the concentration-time curve (AUC). The results were shown in FIG. 17 and Table 5.

TABLE 5
In vivo pharmacokinetic parameters.

| Parameter | Unit | Oral | IV |
|---|---|---|---|
| Dosage | mg/kg | 50 | 1 |
| AUC-(area) | ng-min/ml | 621868 | 104639 |
| [Simpson rule] | | | |
| AUC (0-t) (absolute | ng-min/ml | 539033 | 41934 |
| area) | | | |
| Vd (area)/kg | mL/kg | 20916 | 4812 |
| CL (area)/kg | mL/min/kg | 80 | 10 |
| Elimination half life | min | 180 | 349 |
| Absorption/distribution | min | 21 | 15 |
| half life | | | |
| MRT | min | 289 | 485 |
| Cmax | ng/mL | 1922 | — |
| Tmax | min | 120 | — |
| Bioavailability | % | 11.89% | — |

From the above test results, it can be seen that the novel derivatives derived from 3-aminobenzamide by the preparation methods of the present invention had cytotoxicity/potent antibacterial activity against different bacterial strains (such as Gram positive and Gram negative), cancer cells (LCC6 and LCC6MDR) as well as mouse peritoneal fibroblasts (L929); had low toxicity on mouse fibroblasts; and interacted with the cell division protein FtsZ. We also demonstrated that such derivatives in combination with clinically used β-lactam antibiotics exhibited highly synergic antibacterial activity against clinically isolated community associated S. aureus (USA300) and other MRSA strains with fractional inhibitory concentration index as low as 0.01. Pharmacokinetics study revealed that one of the 3-aminobenzamide derivatives, namely compound F332, at oral dosage of 50 mg/Kg body weight was orally bioavailable at about 12% bioavailability. The derivatives of the present invention can be used in the manufacture of medicaments for the treatment of methicillin-resistant staphylococcal infections and can be used clinically as β-lactam antibiotic adjuvants for the treatment of methicillin-resistant staphylococcal infections by oral route.

1. A 3-aminobenzamide derivative as a β-lactam antibiotic adjuvant, characterized in that it is a compound of the following chemical formula (I), 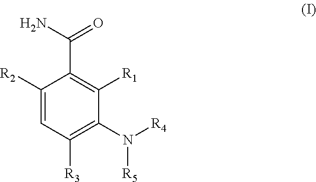
wherein:
R₁, R₂ and R₃ each independently are hydrogen, fluorine or bromine;
when R₄ and R₅ are different, R₄ is hydrogen, methyl, ethyl or fluorobenzyl, and R₅ is C₄-C₁₀ alkyl, alkoxyalkyl, C₄-C₁₀ alkenyl, fluorobenzyl, benzyl substituted by alkoxy, or acyl; when R₄ and R₅ are the same, R₄ and R₅ are both CH₂—(CH₂)ₙ—CH₂, and n=2 or 3.

2. The derivative according to claim 1, characterized in that R₁, R₂, R₃ and R₄ each independently are hydrogen, and R₅ is octyl; R₁, R₂, R₃ and R₄ each independently are hydrogen, and R₅ is nonyl; R₁, R₂ and R₃ each independently are hydrogen, R₄ is methyl, and R₅ is octyl; R₁, R₂ and R₃ each independently are hydrogen, R₄ is methyl, and R₅ is nonyl; R₁ and R₃ each independently are hydrogen, R₂ is F, R₄ is hydrogen, and R₅ is nonyl; or R₁ and R₃ each independently are hydrogen, R₂ and R₄ each are F, and R₅ is nonyl.

3-7. (canceled)

8. A process for preparing the derivative according to claim 1, characterized in that it comprises the steps of:
(a) mixing the starting material A with the starting material B and potassium carbonate in acetonitrile and stirring well to form a reaction mixture, heating the reaction mixture to reflux for 4 hours, and then refining to obtain a product; wherein the starting material A is 3-aminobenzamide, 2-fluoro-5-aminobenzamide or 2,4-difluoro-5-aminobenzamide; the starting material B is 1-bromononane or 1-bromooctane;
(b) when the product obtained in step (a) is 3-(nonylamino)benzamide or 3-(octylamino)benzamide, stirring the product well with dimethyl sulfate and potassium carbonate in acetonitrile, heating to reflux for 12 hours, and then refining to obtain the product.

9. The derivative according to claim 1, characterized in that R₁ and R₂ are F, R₃ and R₄ each are hydrogen, and R₅ is octyl; R₁ and R₂ are F, R₃ and R₄ each are hydrogen, and R₅ is heptyl; R₁ and R₂ are F, R₃ and R₄ each are hydrogen, and R₅ is 3,7-dimethyl-2,6-octadien-1-yl; R₁ and R₂ are F, R₃ and R₄ each are hydrogen, and R₅ is decyl; R₁ and R₂ are F, R₃ is bromine, R₄ is hydrogen, and R₅ is nonyl; or R₁ and R₂ are F, R₃ is hydrogen, R₄ is ethyl, and R₅ is octyl.

10. The derivative according to claim 1, characterized in that R₁ and R₂ are F, R₃ and R₄ each are hydrogen, and R₅ is nonyl.

11. (canceled)

12. (canceled)

13. The derivative according to claim 1, characterized in that R₁ and R₂ are F, R₃ and R₄ each are hydrogen, and R₅ is 2-nonenyl.

14. The derivative according to claim 1, characterized in that R₁ and R₂ are F, R₃ and R₄ each are hydrogen, and R₅ is (4′-butoxy)butyl.

15. (canceled)

16. (canceled)

17. The derivative according to claim 1, characterized in that R₁ and R₂ are F, R₃ is hydrogen, R₄ is methyl, and R₅ is octyl.

18. (canceled)

19. The derivative according to claim 1, characterized in that R₁ and R₂ are F, R₃ is hydrogen, R₄ is methyl, and R₅ is nonyl.

20. A process for preparing the derivative according to claim 1, characterized in that it comprises the steps of:
(a) mixing the starting material 2,6-difluoro-3-aminobenzamide with a brominated alkane and potassium carbonate in acetonitrile and stirring well to form a reaction mixture, heating the reaction mixture to reflux for 4 hours, and then refining to obtain a product; wherein the brominated alkane is 1-bromononane, 1-bromooctane, 1-bromodecane, geranyl bromide, (Z)-1-bromo-2-nonene or 1-bromoheptane;
(b) brominating the 2,6-difluoro-3-(nonylamino)benzamide obtained by the reaction of step (a) and stirring at room temperature for 12 hours, and then refining to obtain 4-bromo-2,6-difluoro-3-(nonylamino)benzamide;
(c) further alkylating the 2,6-difluoro-3-(octylamino)benzamide obtained from step (a) using methyl iodide or bromoethane, mixing with potassium carbonate in acetonitrile with stirring and hating under reflux for 14 hours, and then refining to obtain 2,6-difluoro-3-(methyl(octyl)amino)benzamide or 3-(ethyl(octyl)amino)-2,6-difluorobenzamide;
(d) alkylating the 2,6-difluoro-3-(nonylamino)benzamide obtained from step (a) using dimethyl sulfate, mixing with potassium carbonate in acetonitrile with stirring and heating under reflux for 12 hours, and then refining to obtain 2,6-difluoro-3-(methyl(nonyl)amino)benzamide.

21. The derivative according to claim 1, characterized in that R₁ and R₂ are F, R₃ and R₄ each are hydrogen, and R₅ is nonanamido; R₁ and R₂ are F, R₃ and R₄ each are hydrogen, and R₅ is pyrrolidine; R₁ and R₂ are F, R₃ and R₄ each are hydrogen, and R₅ is piperidine; R₁ and R₂ are F, R₃ and R₄ each are hydrogen, and R₅ is 3′,4′-difluorobenzyl; R₁ and R₂ are F, R₃ is hydrogen, R₄ is 3′,4′-difluorobenzyl, and R₅ is 3′,4′-difluorobenzyl; R₁ and R₂ are F, R₃ and R₄ each are hydrogen, and R₅ is 2′,4′-difluorobenzyl; R₁ and R₂ are F, R₃ is hydrogen, and R₄ and R₅ each respectively are 2′,4′-difluorobenzyl; R₁ and R₂ are F, R₃ and R₄ each are hydrogen, and R₅ is 2′,6′-difluorobenzyl; or R₁ and R₂ are F, R₃ is hydrogen, and R₄ and R₅ each are 2′,6′-difluorobenzyl.

22-29. (canceled)

30. A process for preparing the derivative according to claim 1, characterized in that it comprises the steps of:
(a) dissolving the starting materials 2,6-difluoro-3-aminobenzamide and nonanoyl chloride in pyridine and dichloromethane at 0° C. and allowing to react for 4 hours, and refining to obtain 2,6-difluoro-3-nonylaminobenzamide;
(b) adding potassium carbonate to each of the starting materials 2,6-difluoro-3-aminobenzamide and 1,4-dibromobutane or 1,5-dibromopentane in acetonitrile, stirring well, reacting for 4 hours, and then refining to obtain 2,6-difluoro-3-(pyrrolidin-1-yl)benzamide and 2,6-difluoro-3-(piperidin-1-yl)benzamide, respectively;
(c) heating the starting material 2,6-difluoro-3-aminobenzamide and the starting material C and potassium carbonate in acetonitrile to reflux for 4 hours, and refining to obtain 3-(3′,4′-difluorobenzylamino)-2,6-difluorobenzamide or 3-(bis(3′,4′-difluorobenzyl)amino)-2,6-difluorobenzamide, 3-(2′,4′-difluorobenzylamino)-2,6-difluorobenzamide or 3-(bis(2′,4′-difluorobenzyl)amino)-2,6-difluorobenzamide, and 3-(2′,6′-difluorobenzylamino)-2,6-difluorobenzamide or 3-(bis(2′,6′-difluorobenzyl)amino)-2,6-difluorobenzamide, respectively; wherein the starting material C is 3,4-difluorobenzyl bromide, 2,4-difluorobenzyl bromide or 2,6-difluorobenzyl bromide.

31. The derivative according to claim 1, characterized in that R₁ and R₂ are F, R₃ and R₄ each are hydrogen, and R₅ is 3′-butoxybenzyl; R₁ and R₂ are F, R₃ and R₄ each are hydrogen, and R₅ is 3′-sec-butoxy)benzyl; R₁ and R₂ are F, R₃ and R₄ each are hydrogen, and R₅ is 3′-(pentyloxy)benzyl; or R₁ and R₂ are F, R₃ and R₄ each are hydrogen, and R₅ is 3′-((4″-(trifluoromethyl)benzyl)oxy)benzyl.

32-34. (canceled)

35. A process for preparing the derivative according to claim 1, characterized in that it comprises the steps of:
(a) reacting the starting material 2,6-difluoro-3-aminobenzamide with individual corresponding benzaldehydes and the catalyst p-toluenesulfonic acid (pTsOH) in methanol, and stirring the mixture at room temperature for 2 hours;
(b) then adding sodium cyanoborohydride in portions, mixing and stirring for 12 hours, and refining to obtain the derivative according to any one of claims 31 to 34, respectively; wherein the benzaldehydes are 3-butoxybenzaldehyde, 3-(pentyloxy)benzaldehyde, 3-(sec-butoxy)benzaldehyde or 3-((4′-(trifluoromethyl)benzyl)oxy)benzaldehyde.

36-37. (canceled)

38. A method of treating a methicillin-resistant staphylococcal infection in a patient in need thereof, comprising the steps of administering a derivative of claim 1 and optionally a β-lactam.
